(19) United States
(12) Patent Application Publication
(10) Pub. No.: US 2016/0126469 A1
(43) Pub. Date: May 5, 2016

(54) MATERIAL FOR ORGANIC ELECTROLUMINESCENT DEVICE AND ORGANIC ELECTROLUMINESCENT DEVICE INCLUDING THE SAME

(71) Applicant: SAMSUNG DISPLAY CO., LTD., Yongin-si (KR)

(72) Inventor: Hiromi Nakano, Yokohama (JP)

(21) Appl. No.: 14/925,862

(22) Filed: Oct. 28, 2015

(30) Foreign Application Priority Data
Oct. 29, 2014 (JP) 2014-220102

(51) Int. Cl. H01L 51/00; C07D 307/91

(52) U.S. Cl. CPC H01L 51/0061; C07D 307/91; H01L 51/006; H01L 51/5056

(57) ABSTRACT

Provided are a material for an organic electroluminescent device capable of being driven at a low voltage and having high emission efficiency. An organic electroluminescent device includes the same. An embodiment of the material for an organic electroluminescent device according to the present disclosure is represented by Formula 1. The substituents of Formula 1 are as described herein.

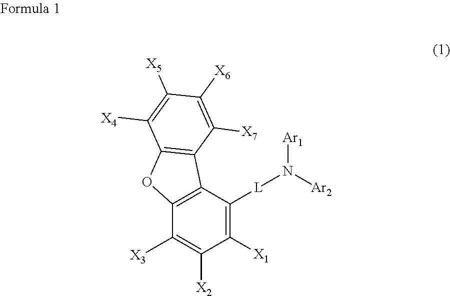

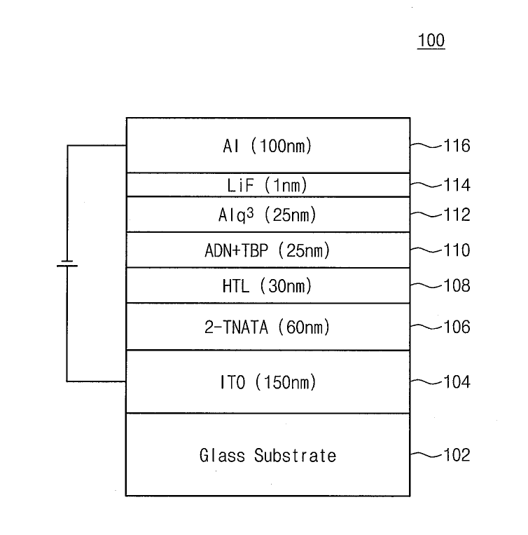

### CROSS-REFERENCE TO RELATED APPLICATION

This patent application claims priority to and the benefit of Japanese Patent Application No. 2014-220102, filed on Oct. 29, 2014, the entire contents of which are hereby incorporated by reference.

### BACKGROUND

Embodiments of the present disclosure relate to a material for an organic electroluminescent device and an organic electroluminescent device including the same. For example, embodiments of the present disclosure relate to a material for an organic electroluminescence device driven at a low voltage and exhibiting high emission efficiency in a blue emission region, and an organic electroluminescence device including the same

In recent years, organic electroluminescent (EL) displays, which are one type or kind of image display, have been actively developed. Unlike a liquid crystal display and the like, the organic EL display is referred to as a self-luminescent display which recombines holes and electrons injected from a positive electrode and a negative electrode in an emission layer to thus emit light from a luminescent material including an organic compound in the emission layer, thereby performing display.

An example of an organic electroluminescent device (organic EL device) is an organic EL device which includes a positive electrode, a hole transport layer disposed on the positive electrode, an emission layer disposed on the hole transport layer, an electron transport layer disposed on the emission layer, and a negative electrode disposed on the electron transport layer. Holes injected from the positive electrode are injected via the hole transport layer into the emission layer. Meanwhile, electrons are injected from the negative electrode, and then injected via the electron transport layer into the emission layer. The holes and the electrons injected into the emission layer are recombined to generate excitons in the emission layer. The organic EL device emits light by using light generated by deactivated radiation produced during the transition of the excitons. Also, the organic EL device is not limited to the above-described configuration but may be changed in various forms.

In the application of the organic EL device in a display apparatus, the low driving voltage and high efficiency of the organic EL device are beneficial or required. For example, the driving voltage is high and the emission efficiency is insufficient in a blue emission region and in a green emission region of the organic EL when compared to those in a red emission region. To realize the driving at a low voltage and the high efficiency of the organic EL device, the normalization and the stabilization of a hole transport layer have been examined. As a useful material for the increase of the life of an organic EL device, an amine compound having a dibenzofuran group is suggested, such as an amine derivative including fluorene and dibenzofuran, an amine derivative having a terphenyl group and dibenzofuran, a polyamine in which an amine part includes 2 to 10 dibenzofuran groups, an amine having carbazole and dibenzofuran, and a dibenzofuran derivative.

In addition, an anthracene derivative having dibenzofuran and amine as substituents may also be used. A material for an organic EL device having an amino group making a direct linkage with dibenzofuran may be used. Dibenzofuran having a substituent including an amine at position 2 may also be used. An amine derivative having dibenzofuran including triphenylene and a carbazole connecting group may be used. An amine derivative in which amine makes a direct linkage at position 1, and a carbazole group is substituted with a dibenzofuran skeleton may also be used.

A compound including a terphenyl group or a fluorene ring structure may unsuitably or undesirably induce an increase of an evaporation temperature and thermal decomposition of a material during processing. In addition, the compound may increase electron transport properties, and when applied in an electron blocking layer, may not improve the life and emission efficiency of an organic EL device at the same time.

However, the organic EL device using the material is difficult to say to have a suitably or sufficiently low driving voltage and high emission efficiency, and an organic EL device having a lower driving voltage and higher emission efficiency is desirable or required as of now. For example, since the emission efficiency of the organic EL device is low in a blue emission region and a green emission region when compared with a red emission region, the increase of the emission efficiency is desirable or required. The development of a novel material to realize the driving at a low voltage and higher efficiency of an organic EL device is desirable or necessary.

### SUMMARY

Aspects of embodiments of the present disclosure address the above-mentioned defects by providing a material for an organic electroluminescent device driven at a low voltage and having high emission efficiency, and an organic electroluminescent device including the same.

An embodiment of the present disclosure provides a material for an organic EL device represented by the following Formula 1.

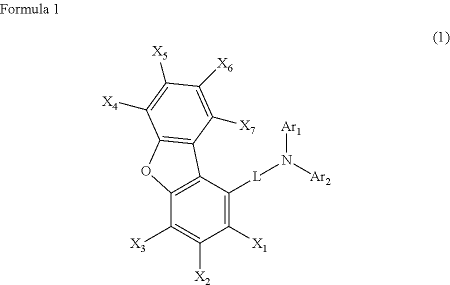

In the above Formula 1, X₁-X₇ are each independently a hydrogen atom, a deuterium atom, a halogen atom, an alkyl group having 1 to 15 carbon atoms, a substituted or unsubstituted aryl group having 6 to 30 carbon atoms for forming a ring, or a substituted or unsubstituted heteroaryl group having 5 to 30 carbon atoms for forming a ring, Ar₁ and Ar₂ are each an aryl group having 6 to 30 carbon atoms for forming a ring, or a substituted or unsubstituted heteroaryl group having 1 to 30 carbon atoms for forming a ring, and Ar₁ and Ar₂ each does not include the same structure as a dibenzofuran group including L in Formula 1, and L is a divalent connecting group having a triplet energy gap of 2.5 eV and above.

Since the material for an organic EL device according to an embodiment is an amine derivative having 1-dibenzofurane group and a 1-substituted dibenzofuran part, energy gap may increase, and energy transfer to an adjacent layer may be restrained when the material is applied in an organic EL device. Thus, the driving at a low voltage and high emission efficiency may be realized, and remarkable effects may be obtained, for example, in a blue emission region and a green emission region.

In an embodiment, L may be a divalent group selected from a substituted or unsubstituted arylene group or heteroarylene group represented by the following Formula 2, and n may be an integer from 1 to 3.

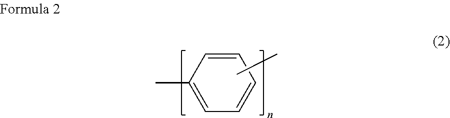

In the material for an organic EL device according to an embodiment of the present disclosure, a 1-substituted dibenzofuran part is combined with the nitrogen atom of amine using the connecting group, energy gap may increase, and energy transfer to an adjacent layer may be restrained. Thus, the driving at a low voltage and high emission efficiency may be realized.

In an embodiment of the present disclosure, an organic EL device includes one of the materials for an organic EL device in an emission layer.

Since the organic EL device according to an embodiment includes an amine derivative having 1-dibenzofurane group and a 1-substituted dibenzofuran part in the emission layer, energy gap may increase, and energy transfer to an adjacent layer may be restrained. Thus, the driving at a low voltage and high emission efficiency may be realized, and remarkable effects may be obtained, for example, in a blue emission region and a green emission region.

In an embodiment of the present disclosure, an organic EL device includes one of the materials for an organic EL device in a layer of stacking layers disposed between an emission layer and an anode.

Since the organic EL device according to an embodiment includes an amine derivative having 1-dibenzofurane group and a 1-substituted dibenzofuran part in the layer of stacking layers disposed between the emission layer and the anode, energy gap may increase, and energy transfer to an adjacent layer may be restrained. Thus, the driving at a low voltage and high emission efficiency may be realized, and remarkable effects may be obtained, for example, in a blue emission region and a green emission region.

### BRIEF DESCRIPTION OF THE FIGURES

The accompanying drawing is included to provide a further understanding of embodiments of the present disclosure, and is incorporated in and constitutes a part of this specification. The drawing illustrates an exemplary embodiment of the present disclosure and, together with the description, serves to explain principles of embodiments of the present disclosure.

The accompanying drawing is a schematic diagram illustrating an organic EL device according to an embodiment of the present disclosure.

### DETAILED DESCRIPTION

According to embodiments of the present disclosure, an energy gap may increase, energy transfer to an adjacent layer may be restrained, and the driving at a low voltage and high emission efficiency of an organic EL device may be realized by using an amine derivative having a 1-dibenzofuran group and a 1-substituted dibenzofuran part instead of an amine compound substituted at position 2 of dibenzofuran as used in other amine derivatives.

Hereinafter, a material for an organic EL device and an organic EL device including the same according to embodiments of the present disclosure will be described in more detail with reference to the accompanying drawing. The material for an organic EL device and the organic EL device including the same according to embodiments of the present disclosure may, however, be embodied in different forms and should not be construed as limited to the embodiments set forth herein. In the description and drawings, elements having substantially the same function are designated by the same reference numerals, and repeated explanation thereof will not be provided. In the drawings, the relative sizes of elements, layers, and regions may be exaggerated for clarity.

The material for an organic EL device according to embodiments of the present disclosure includes an amine derivative including a 1-dibenzofuran group and a 1-substituted dibenzofuran part and is represented by the following Formula 1.

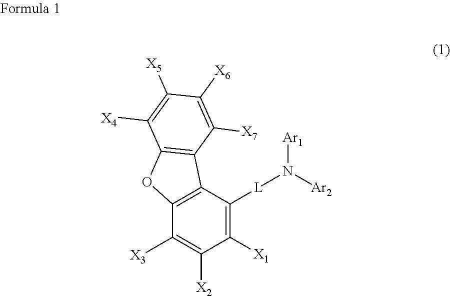

In Formula 1, X₁-X₇ are each independently a hydrogen atom, a deuterium atom, a halogen atom, an alkyl group having 1 to 15 carbon atoms, a substituted or unsubstituted aryl group having 6 to 30 carbon atoms for forming a ring, or a substituted or unsubstituted heteroaryl group having 5 to 30 carbon atoms for forming a ring. Ar₁ and Ar₂ are each an aryl group having 6 to 30 carbon atoms for forming a ring, or a heteroaryl group having 1 to 30 carbon atoms for forming a ring, and Ar₁ and Ar₂ do not include the same structure as a dibenzofuran group including L in Formula 1. In addition, L is a divalent connecting group having a triplet energy gap of 2.5 eV and above (e.g., a triplet energy gap of 2.5 eV or more).

As the alkyl group having 1 to 15 carbon atoms used as X₁ to X₇, examples thereof may include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, etc., without limitation.

Examples of the substituted or unsubstituted aryl group having 6 to 30 carbon atoms for forming a ring, used as X₁ to X₇ may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc., without limitation.

Examples of the substituted or unsubstituted heteroaryl group having 5 to 30 carbon atoms for forming a ring, used as X₁ to X₇ may include a benzothiazolyl group, a thiophenyl group, a thienothiophenyl group, a thienothienothiophenyl group, a benzothiophenyl group, a benzofuryl group, a dibenzothiophenyl group, an N-arylcarbazolyl group, an N-heteroarylcarbazolyl group, an N-alkylcarbazolyl group, a phenoxazyl group, a phenothiazyl group, a pyridyl group, a pyrimidyl group, a triazile group, a quinolinyl group, a quinoxalyl group, etc., without limitation.

Examples of the substituted or unsubstituted aryl group having 6 to 30 carbon atoms for forming a ring, used as Ar₁ and Ar₂ may include a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a pyrenyl group, a benzofluoranthenyl group, a chrysenyl group, etc., without limitation.

Examples of the heteroaryl group having 1 to 30 carbon atoms for forming a ring, used as Ar₁ and Ar₂ may include a dibenzofuran group, a dibenzothiophene group, a carbazolyl group, a dibenzosilole group, etc., without limitation.

As described above, Ar₁ and Ar₂ do not include the same structure as the dibenzofuran group including L in Formula 1. For example, in some embodiments, the material represented by Formula 1 is not symmetric about L. In the case that Ar₁ or Ar₂ includes the same structure as the dibenzofuran group including L in Formula 1, the symmetry of an amine compound may increase, and the amorphous properties of the material for an organic EL device may be deteriorated. For example, light transmittance of an organic EL device may be deteriorated with the increase of crystallinity (e.g., with the increase of crystallinity of material represented by Formula 1).

In addition, in the material for an organic EL device according to the present disclosure, L is a divalent group having the energy gap of triplet of 2.5 eV and above (e.g., the divalent group of L may have a triplet energy gap of 2.5 eV or more). If the energy gap of the triplet of the energy level of the connecting group L (e.g., the triplet energy gap of L) decreases to less than 2.5 eV, energy transfer in the organic EL device may be easily conducted, and emission efficiency may tend unsuitably or undesirably decrease.

In some embodiments, the connecting group L having the above-identified features is a divalent group selected from a substituted or unsubstituted arylene group or heteroarylene group. For example, L may be a divalent group selected from a substituted or unsubstituted arylene group represented by Formula 2. In Formula 2, at least one of the ring carbons may be substituted with a heteroatom to form a substituted or unsubstituted heteroarylene group. In some embodiments, n in Formula 2 is an integer from 1 to 3. In the case that n is 4 or above, the molecular weight of the material for an organic EL device may be too high (e.g., unsuitably or undesirably high), and this material may not be suitable or appropriate in a deposition process.

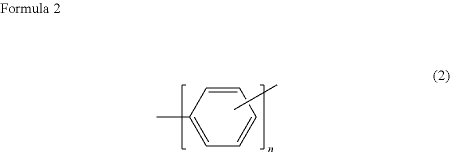

The energy gap may increase, energy transfer to an adjacent layer may be restrained, and the driving at a low voltage and high emission efficiency may be realized by using an amine derivative having a 1-dibenzofuran group and a 1-substituted dibenzofuran part in the material for an organic EL device according to embodiments of the present disclosure. For example, remarkable effects may be obtained in a blue emission region and a green emission region.

The material for an organic EL device according to the present disclosure may include at least one compound selected from Compounds 1 to 7.

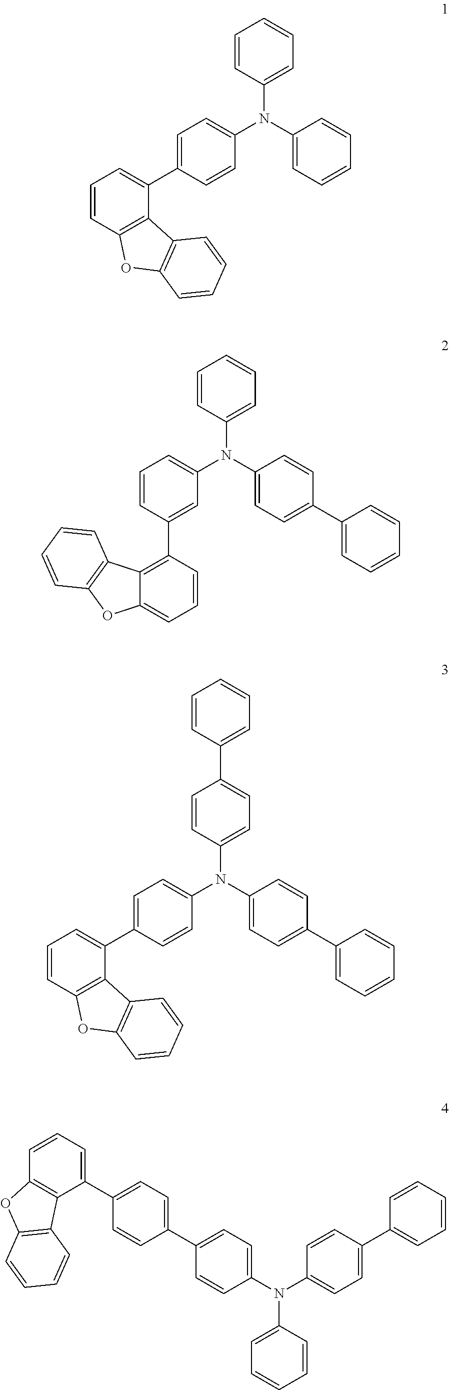 [SMILES: c1ccc(N(c2ccccc2)c2ccc(-c3cccc4oc5ccccc5c34)cc2)cc1 ; c1ccc(-c2ccc(N(c3ccccc3)c3cccc(-c4cccc5oc6ccccc6c45)c3)cc2)cc1 ; c1ccc(-c2ccc(N(c3ccc(-c4ccccc4)cc3)c3ccc(-c4cccc5oc6ccccc6c45)cc3)cc2)cc1 ; c1ccc(-c2ccc(N(c3ccccc3)c3ccc(-c4ccc(-c5cccc6oc7ccccc7c56)cc4)cc3)cc2)cc1]

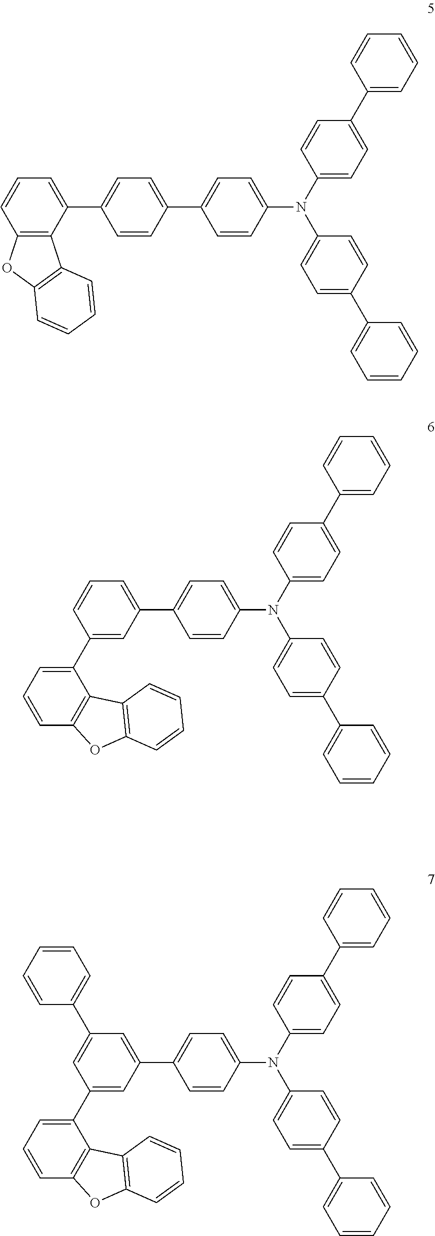 [SMILES: c1ccc(-c2ccc(N(c3ccc(-c4ccccc4)cc3)c3ccc(-c4ccc(-c5cccc6oc7ccccc7c56)cc4)cc3)cc2)cc1 ; c1ccc(-c2ccc(N(c3ccc(-c4ccccc4)cc3)c3ccc(-c4cccc(-c5cccc6oc7ccccc7c56)c4)cc3)cc2)cc1 ; c1ccc(-c2ccc(N(c3ccc(-c4ccccc4)cc3)c3ccc(-c4cc(-c5ccccc5)cc(-c5cccc6oc7ccccc7c56)c4)cc3)cc2)cc1]

The material for an organic EL device according to the present disclosure may include at least one compound selected from Compounds 8 to 12.

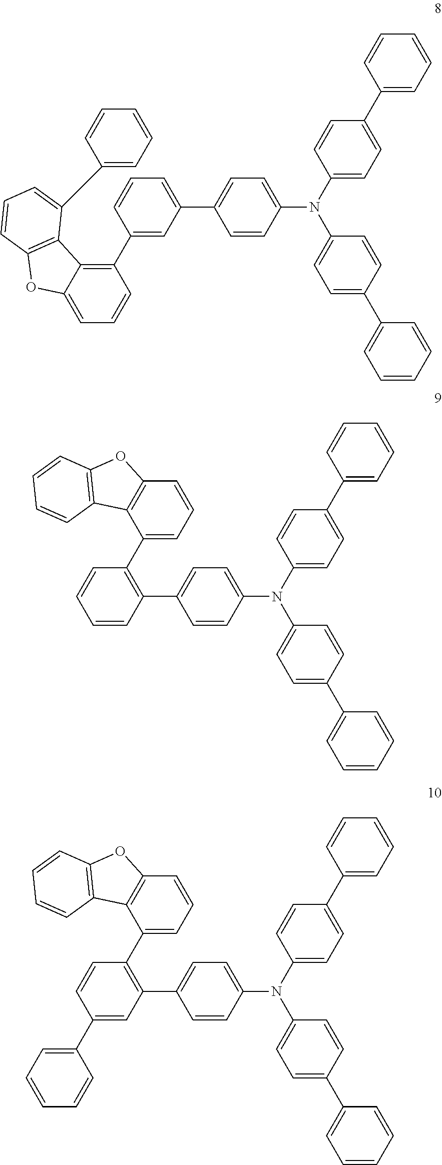

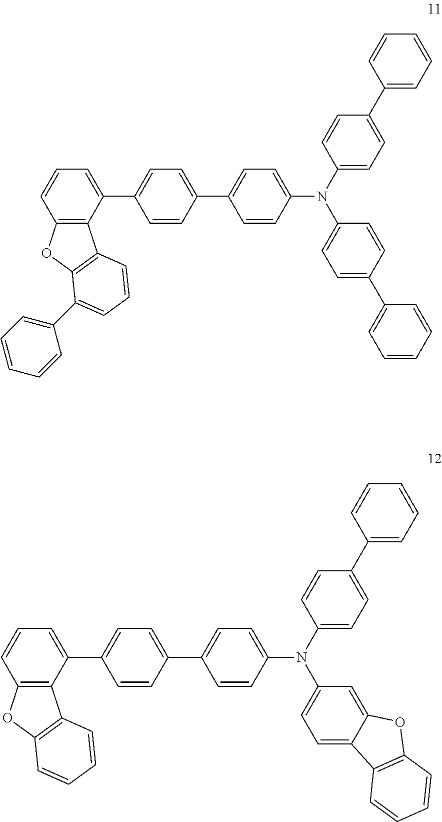 [SMILES: c1ccc(-c2ccc(N(c3ccc(-c4ccccc4)cc3)c3ccc(-c4ccc(-c5cccc6oc7c(-c8ccccc8)cccc7c56)cc4)cc3)cc2)cc1 ; c1ccc(-c2ccc(N(c3ccc(-c4ccc(-c5cccc6oc7ccccc7c56)cc4)cc3)c3ccc4c(c3)oc3ccccc34)cc2)cc1]

The material for an organic EL device according to the present disclosure may include at least one compound selected from Compounds 13 to 20.

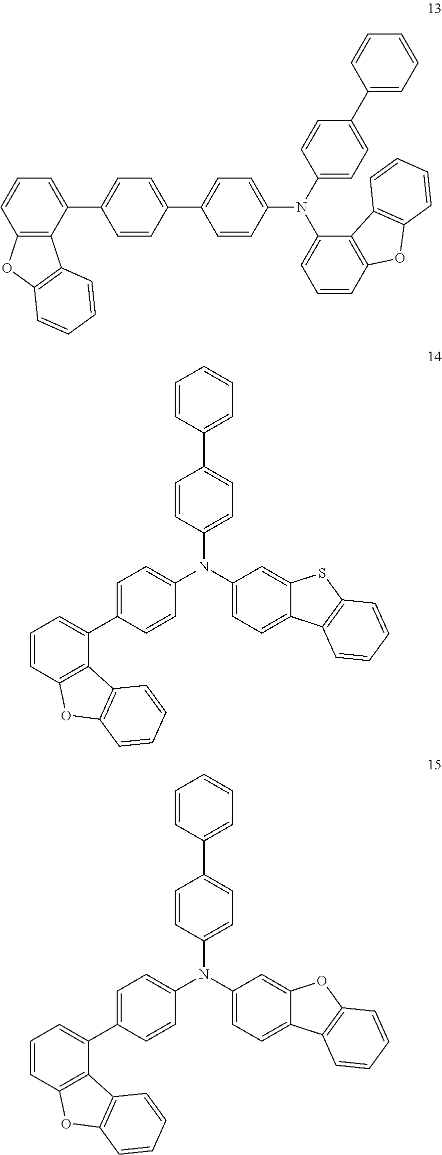 [SMILES: c1ccc(-c2ccc(N(c3ccc(-c4ccc(-c5cccc6oc7ccccc7c56)cc4)cc3)c3cccc4oc5ccccc5c34)cc2)cc1 ; c1ccc(-c2ccc(N(c3ccc(-c4cccc5oc6ccccc6c45)cc3)c3ccc4c(c3)sc3ccccc34)cc2)cc1 ; c1ccc(-c2ccc(N(c3ccc(-c4cccc5oc6ccccc6c45)cc3)c3ccc4c(c3)oc3ccccc34)cc2)cc1]

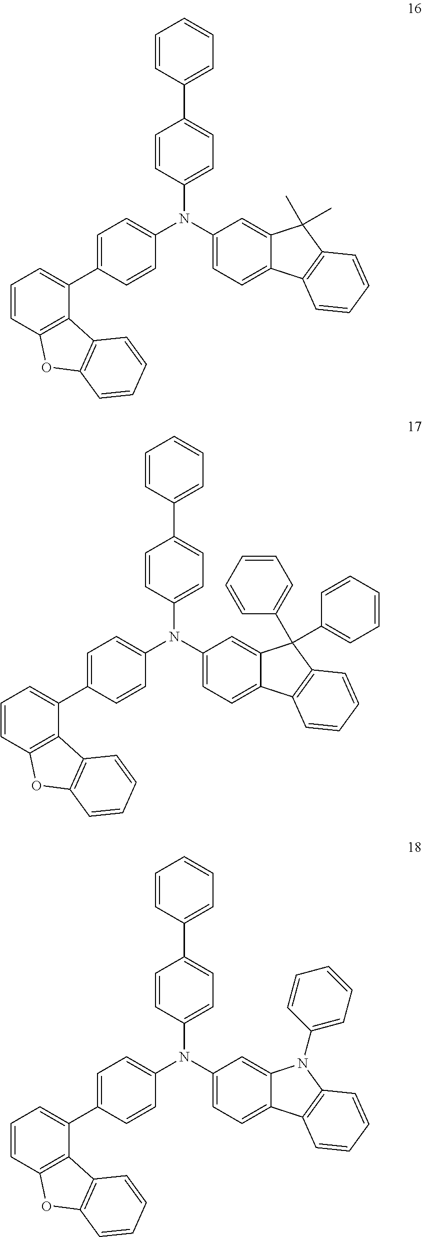 [SMILES: CC1(C)c2ccccc2-c2ccc(N(c3ccc(-c4ccccc4)cc3)c3ccc(-c4cccc5oc6ccccc6c45)cc3)cc21 ; c1ccc(-c2ccc(N(c3ccc(-c4cccc5oc6ccccc6c45)cc3)c3ccc4c(c3)C(c3ccccc3)(c3ccccc3)c3ccccc3-4)cc2)cc1 ; c1ccc(-c2ccc(N(c3ccc(-c4cccc5oc6ccccc6c45)cc3)c3ccc4c5ccccc5n(-c5ccccc5)c4c3)cc2)cc1]

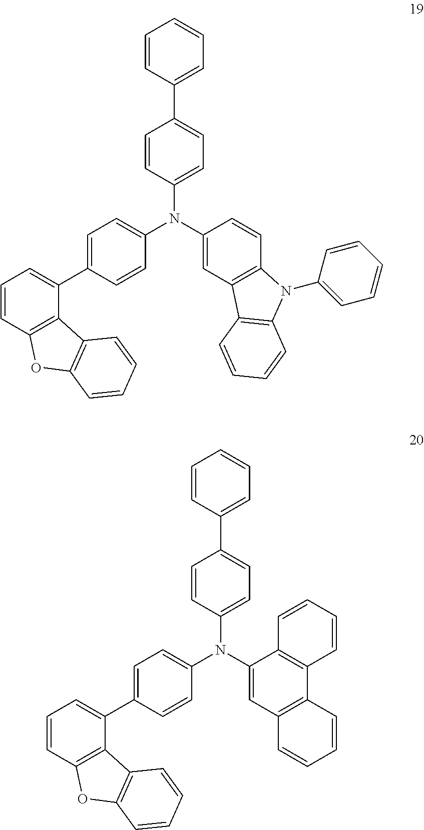 [SMILES: c1ccc(-c2ccc(N(c3ccc(-c4cccc5oc6ccccc6c45)cc3)c3ccc4c(c3)c3ccccc3n4-c3ccccc3)cc2)cc1 ; c1ccc(-c2ccc(N(c3ccc(-c4cccc5oc6ccccc6c45)cc3)c3cc4ccccc4c4ccccc34)cc2)cc1]

The material for an organic EL device according to the present disclosure may include at least one compound selected from Compounds 21 to 26.

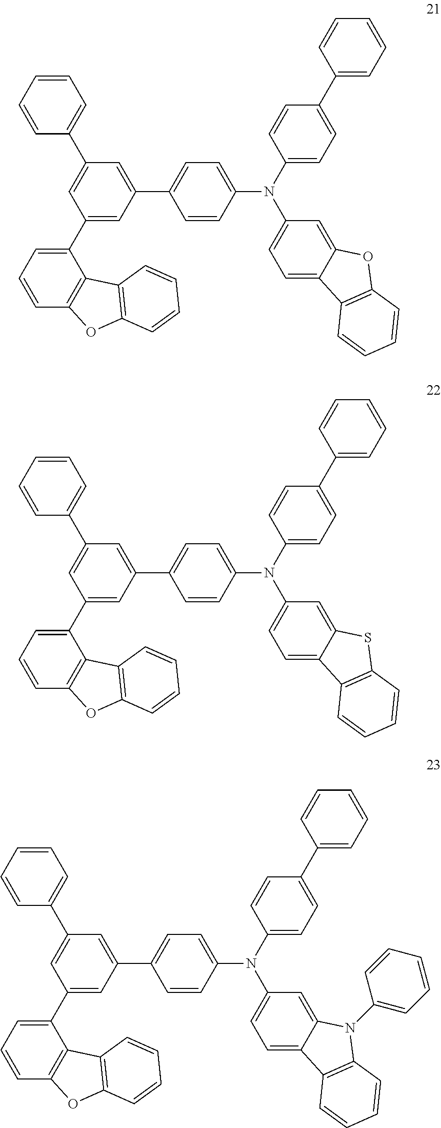

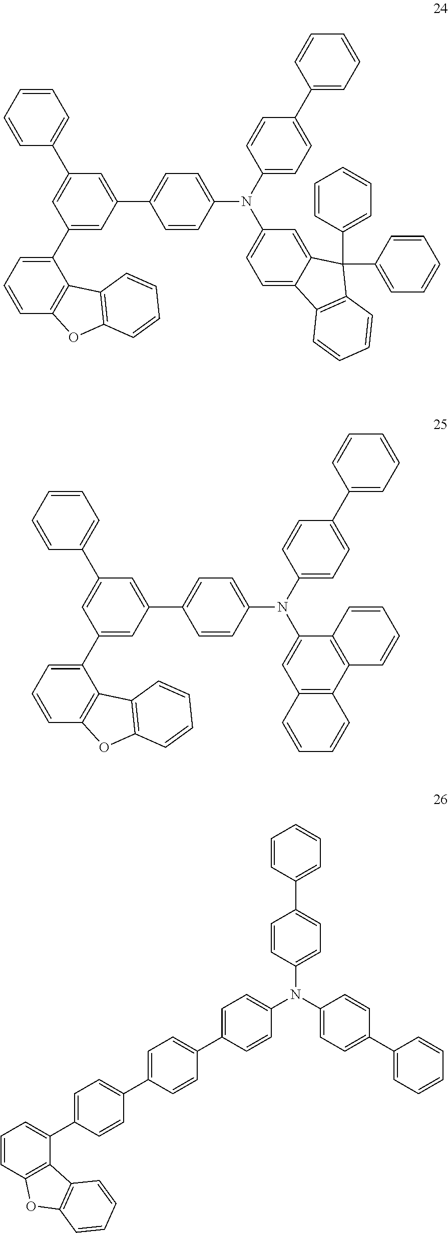 [SMILES: c1ccc(-c2ccc(N(c3ccc(-c4cc(-c5ccccc5)cc(-c5cccc6oc7ccccc7c56)c4)cc3)c3ccc4c(c3)C(c3ccccc3)(c3ccccc3)c3ccccc3-4)cc2)cc1 ; c1ccc(-c2ccc(N(c3ccc(-c4cc(-c5ccccc5)cc(-c5cccc6oc7ccccc7c56)c4)cc3)c3cc4ccccc4c4ccccc34)cc2)cc1 ; c1ccc(-c2ccc(N(c3ccc(-c4ccccc4)cc3)c3ccc(-c4ccc(-c5ccc(-c6cccc7oc8ccccc8c67)cc5)cc4)cc3)cc2)cc1]

The material for an organic EL device according to the present disclosure may include at least one compound selected from Compounds 27 to 32.

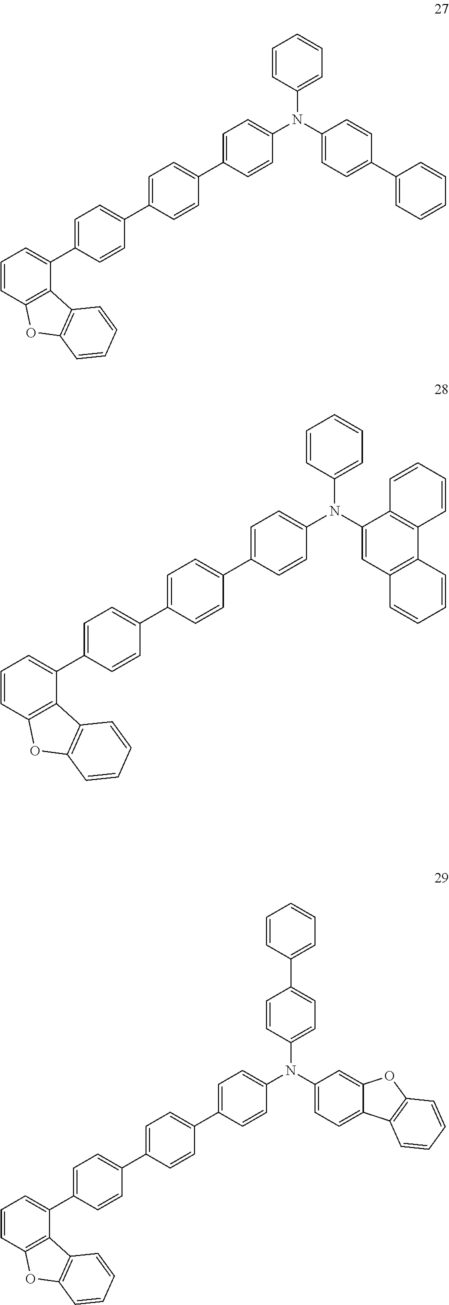 [SMILES: c1ccc(-c2ccc(N(c3ccccc3)c3ccc(-c4ccc(-c5ccc(-c6cccc7oc8ccccc8c67)cc5)cc4)cc3)cc2)cc1 ; c1ccc(N(c2ccc(-c3ccc(-c4ccc(-c5cccc6oc7ccccc7c56)cc4)cc3)cc2)c2cc3ccccc3c3ccccc23)cc1 ; c1ccc(-c2ccc(N(c3ccc(-c4ccc(-c5ccc(-c6cccc7oc8ccccc8c67)cc5)cc4)cc3)c3ccc4c(c3)oc3ccccc34)cc2)cc1]

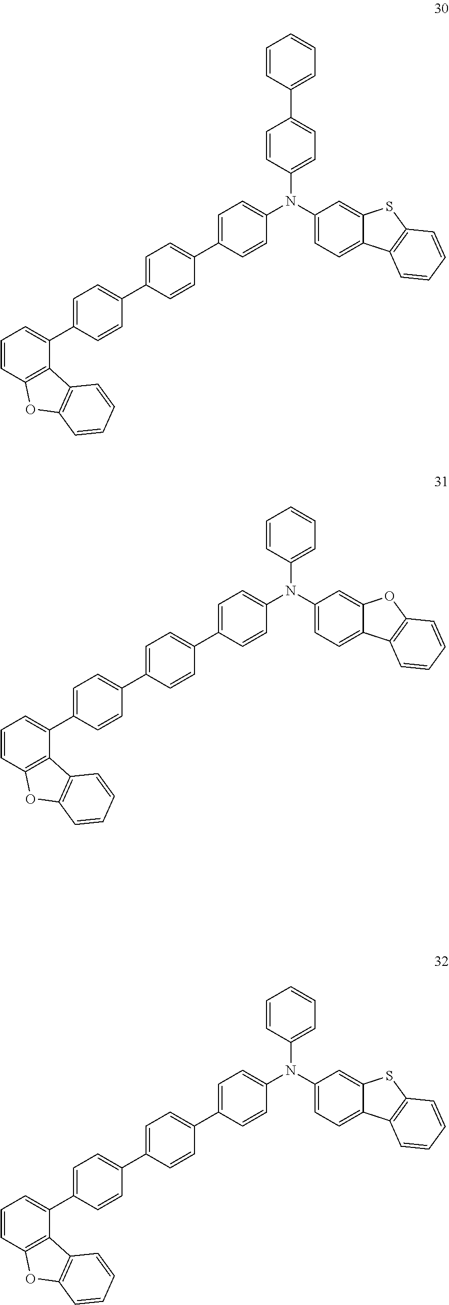 [SMILES: c1ccc(-c2ccc(N(c3ccc(-c4ccc(-c5ccc(-c6cccc7oc8ccccc8c67)cc5)cc4)cc3)c3ccc4c(c3)sc3ccccc34)cc2)cc1 ; c1ccc(N(c2ccc(-c3ccc(-c4ccc(-c5cccc6oc7ccccc7c56)cc4)cc3)cc2)c2ccc3c(c2)oc2ccccc23)cc1 ; c1ccc(N(c2ccc(-c3ccc(-c4ccc(-c5cccc6oc7ccccc7c56)cc4)cc3)cc2)c2ccc3c(c2)sc2ccccc23)cc1]

The material for an organic EL device according to the present disclosure may include at least one compound selected from Compounds 33 to 36.

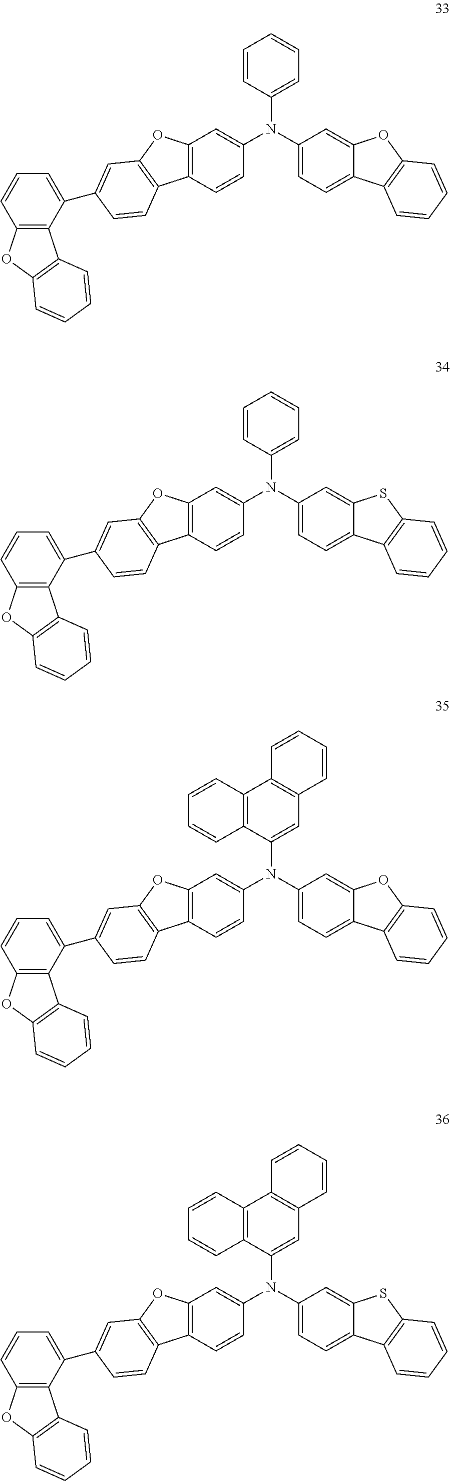 [SMILES: c1ccc(N(c2ccc3c(c2)oc2ccccc23)c2ccc3c(c2)oc2cc(-c4cccc5oc6ccccc6c45)ccc23)cc1 ; c1ccc(N(c2ccc3c(c2)oc2cc(-c4cccc5oc6ccccc6c45)ccc23)c2ccc3c(c2)sc2ccccc23)cc1 ; c1ccc2c(c1)cc(N(c1ccc3c(c1)oc1ccccc13)c1ccc3c(c1)oc1cc(-c4cccc5oc6ccccc6c45)ccc13)c1ccccc12 ; c1ccc2c(c1)cc(N(c1ccc3c(c1)oc1cc(-c4cccc5oc6ccccc6c45)ccc13)c1ccc3c(c1)sc1ccccc13)c1ccccc12]

The material for an organic EL device may be suitably or appropriately used in the emission layer of an organic device. In some embodiments, the material for an organic EL device may be suitably or appropriately used in at least a layer of stacking layers disposed between an emission layer and an anode. Thus, hole transport properties may be improved, and the driving at a low voltage and high efficiency of an organic EL device may be realized.

(Organic EL Device)

An organic EL device using the material for an organic EL device according to embodiments of the present disclosure will be explained. The accompanying drawing is a schematic diagram illustrating an organic EL device 100 according to an embodiment of the present disclosure. The organic EL device 100 may include, for example, a substrate 102, a positive electrode 104, a hole injection layer 106, a hole transport layer 108, an emission layer 110, an electron transport layer 112, an electron injection layer 114 and a negative electrode 116. In an embodiment, the material for an organic EL device according to the present disclosure may be used in an emission layer of an organic EL device. In another embodiment, the material for an organic EL device according to an embodiment of the present disclosure may be used in a layer of stacking layers disposed between an emission layer and a positive electrode.

For example, an embodiment including the material for an organic EL device according to the present disclosure in the hole transport layer 108 will now be explained in more detail. The substrate 102 may be a transparent glass substrate, a semiconductor substrate formed by using silicon, etc., or a flexible substrate of a resin, etc. The positive electrode 104 is disposed on the substrate 102 and may be formed by using indium tin oxide (ITO), indium zinc oxide (IZO), etc. The hole injection layer 106 is disposed on the positive electrode 104 and may include, for example, 4,4′,4″-tris[2-naphthyl(phenyl)amino]triphenylamine (2-TNATA), N,N,N′,N′-tetrakis(3-methylphenyl)-3,3′-dimethylbenzidine (HMTPD), etc. The hole transport layer 108 (HTL) is disposed on the hole injection layer 106, and may be formed by using the material for an organic EL device according to the present disclosure. The emission layer 110 is disposed on the hole transport layer 108 and may be formed using the material for an organic EL device according to the present disclosure. In some embodiments, the emission layer 110 may be formed, for example, by doping a host material including 9,10-di(2-naphthyl)anthracene (AND) with 2,5,8,11-tetra-t-butylperylene (TBP). The electron transport layer 112 is disposed on the emission layer 110 and may be formed using a material including tris(8-hydroxyquinolinato)aluminum (Alq3). The electron injection layer 114 is disposed on the electron transport layer 112 and may be formed by using, for example, a material including lithium fluoride (LiF). The negative electrode 116 is disposed on the electron injection layer 114 and may be formed by using a metal such as Al or a transparent material such as ITO, IZO, etc. The thin layers may be formed by selecting a suitable or appropriate layer forming method such as vacuum deposition, sputtering, diverse coatings, etc. according to the materials used.

In the organic EL device 100 according to an embodiment of the present disclosure, a hole transport layer having high efficiency and long life may be formed by using the material for an organic EL device according to embodiments of the present disclosure. In addition, the material for an organic EL device according to the present disclosure may be applied in an organic EL apparatus of an active matrix type or kind using thin film transistors (TFT).

In addition, since the organic EL device 100 according to an embodiment of the present disclosure includes the material for an organic EL device according to embodiments of the present disclosure in an emission layer or a layer of stacking layers disposed between the emission layer and a positive electrode, the high efficiency and the long life of the organic EL device may be realized.

### Examples

### Preparation Method

The above-described materials for an organic EL device according to embodiments of the present disclosure may be synthesized, for example, as follows. Compound 3 according to an example may be prepared, for example, by the following method.

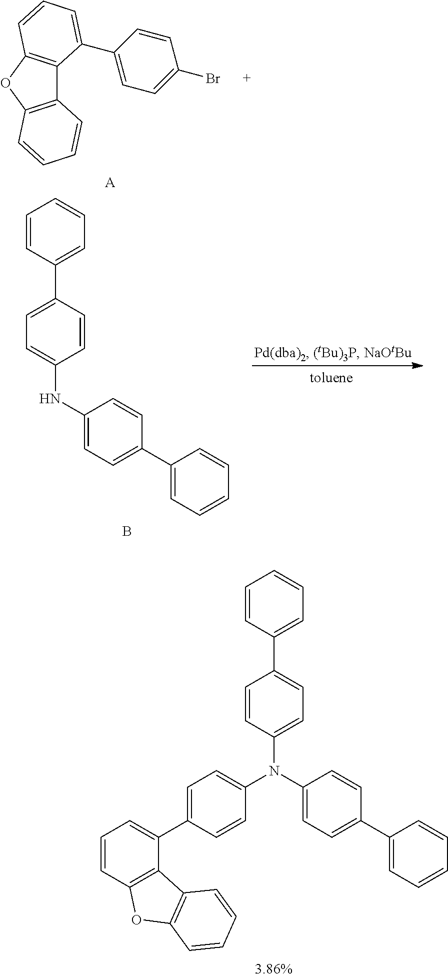

Compound 3 was synthesized by the following procedure. Under an argon atmosphere, 1.50 g of Compound A, 1.90 g of Compound B, 0.11 g of bis(dibenzylideneacetone)palladium(0) (Pd(dba)₂), 0.15 g of tri-tert-butylphosphine ((t-Bu)₃P), 0.54 g of sodium tert-butoxide were added to a 100 ml, three-necked flask, followed by heating and refluxing in 45 ml of a toluene solvent for about 6 hours. After air cooling, water was added, an organic layer was separated, and solvents were distilled. The crude product thus obtained was separated by silica gel column chromatography (using a mixture solvent of dichloromethane and hexane) and recrystallized using a mixture solvent of toluene and hexane to obtain 2.25 g of a target product as a white solid (Yield 86%). In the reaction scheme above, Compound 3 is shown at the right of the reaction scheme at a yield of 86%.

The chemical shift values measured by ¹H NMR were 7.98 (d, 1H), 7.82 (d, 1H), 7.75-7.69 (m, 3H), 7.55-7.31 (m, 24H). In addition, the molecular weight of the target product measured by fast atom bombardment mass spectrometry (FAB-MS) was 564. As a result, the target product was determined as Compound 3.

Compound 7 according to an embodiment may be synthesized, for example, by the following method.

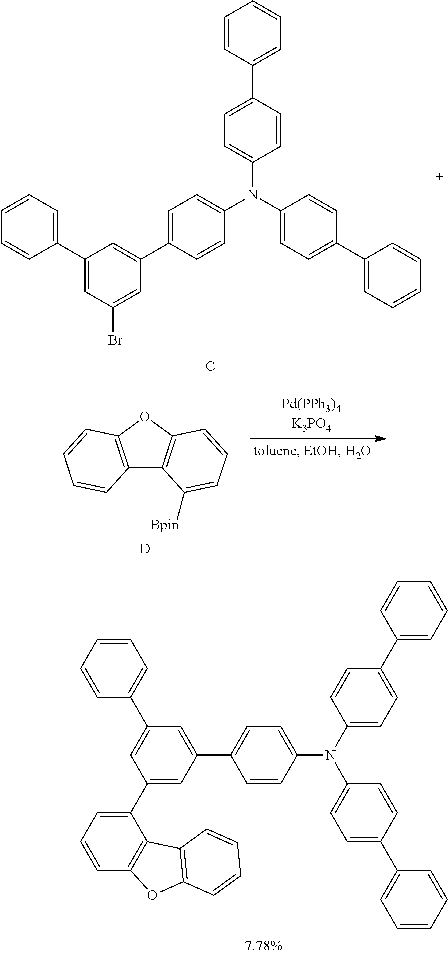

Compound 7 was synthesized by the following procedure. Under an argon atmosphere, 1.2 g of Compound C, 0.35 g of Compound D, 0.11 g of tetrakistriphenylphosphinepalladium(O) (Pd(PPh₃)₄) and 0.15 g of potassium phosphate were added to a 100 ml, three-necked flask, followed by heating and refluxing in 50 ml of a mixture solvent of toluene, ethanol and water for about 6 hours. After air cooling, water was added, an organic layer was separated, and solvents were distilled. The crude product thus obtained was separated by silica gel column chromatography (using a mixture solvent of dichloromethane and hexane) and recrystallized using a mixture solvent of toluene and hexane to obtain 1.05 g of a target product as a white solid (Yield 78%). In the reaction scheme above, Compound 7 is shown at the right of the reaction scheme at a yield of 78%.

The chemical shift values measured by ¹H NMR were 8.04 (s, 3H), 7.98 (d, 1H), 7.82 (d, 1H), 7.75-7.69 (m, 7H), 7.50-7.29 (m, 25H). In addition, the molecular weight of the target product measured by FAB-MS was 715. As a result, the target product was determined as Compound 7.

Compound 17 according to an embodiment may be synthesized, for example, by the following method.

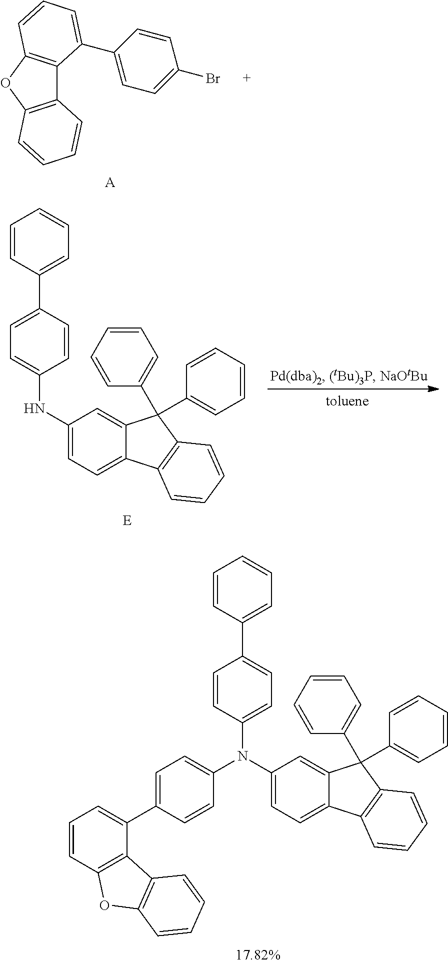

Compound 17 was synthesized by the following procedure. Under an argon atmosphere, 1.50 g of Compound A, 2.3 g of Compound E, 0.15 g of bis(dibenzylideneacetone)palladium(O) (Pd(dba)₂), 0.18 g of tri-tert-butylphosphine ((t-Bu)₃P), 0.48 g of sodium tert-butoxide were added to a 100 ml, three-necked flask, followed by heating and refluxing in 50 ml of a toluene solvent for about 6 hours. After air cooling, water was added, an organic layer was separated, and solvents were distilled. The crude product thus obtained was separated by silica gel column chromatography (using a mixture solvent of dichloromethane and hexane) and recrystallized using a mixture solvent of toluene and hexane to obtain 2.77 g of a target product as a white solid (Yield 82%). In the reaction scheme above, Compound 17 is shown at the right of the reaction scheme at a yield of 82%.

The chemical shift values measured by ¹H NMR were 7.99 (d, 1H), 7.90-7.69 (m, 6H), 7.57-7.18 (m, 30H). In addition, the molecular weight of the target product measured by FAB-MS was 728. As a result, the target product was determined as Compound 17.

Organic EL devices according to Examples 1 to 6 were manufactured using Compounds 3, 7, 17, 21, 26 and 33 as hole transport materials by the above-mentioned manufacturing method. Compounds 3, 7, 17, 21, 26, and 33 are shown below.

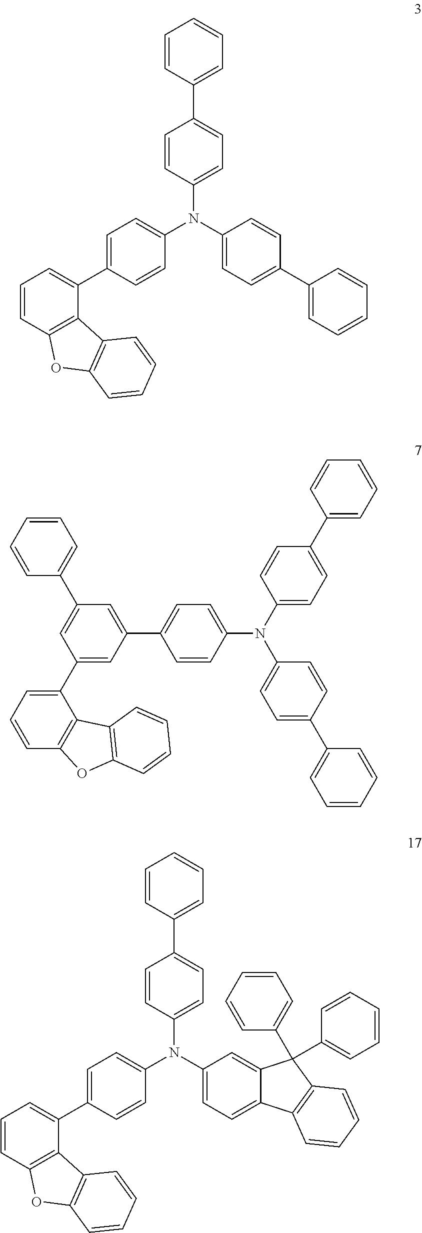 [SMILES: c1ccc(-c2ccc(N(c3ccc(-c4ccccc4)cc3)c3ccc(-c4cccc5oc6ccccc6c45)cc3)cc2)cc1 ; c1ccc(-c2ccc(N(c3ccc(-c4ccccc4)cc3)c3ccc(-c4cc(-c5ccccc5)cc(-c5cccc6oc7ccccc7c56)c4)cc3)cc2)cc1 ; c1ccc(-c2ccc(N(c3ccc(-c4cccc5oc6ccccc6c45)cc3)c3ccc4c(c3)C(c3ccccc3)(c3ccccc3)c3ccccc3-4)cc2)cc1]

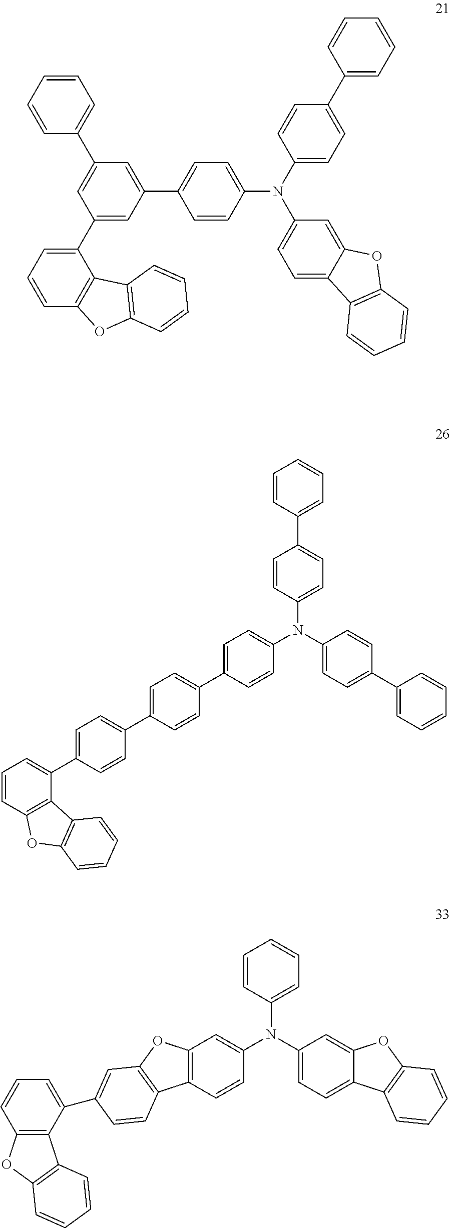 [SMILES: c1ccc(-c2ccc(N(c3ccc(-c4cc(-c5ccccc5)cc(-c5cccc6oc7ccccc7c56)c4)cc3)c3ccc4c(c3)oc3ccccc34)cc2)cc1 ; c1ccc(-c2ccc(N(c3ccc(-c4ccccc4)cc3)c3ccc(-c4ccc(-c5ccc(-c6cccc7oc8ccccc8c67)cc5)cc4)cc3)cc2)cc1 ; c1ccc(N(c2ccc3c(c2)oc2ccccc23)c2ccc3c(c2)oc2cc(-c4cccc5oc6ccccc6c45)ccc23)cc1]

In addition, organic EL devices according to Comparative Examples 1 to 5 were manufactured using the following Compounds 37 to 41 as hole transport materials.

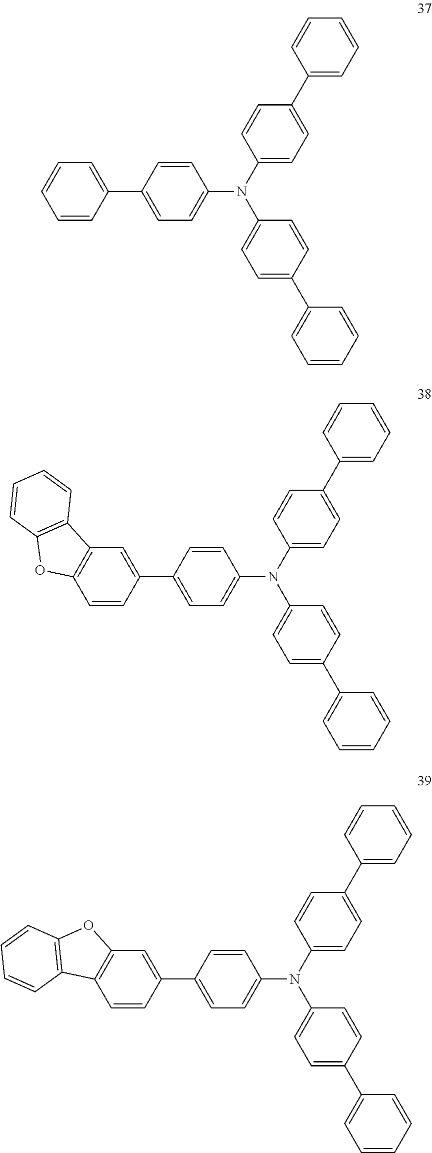

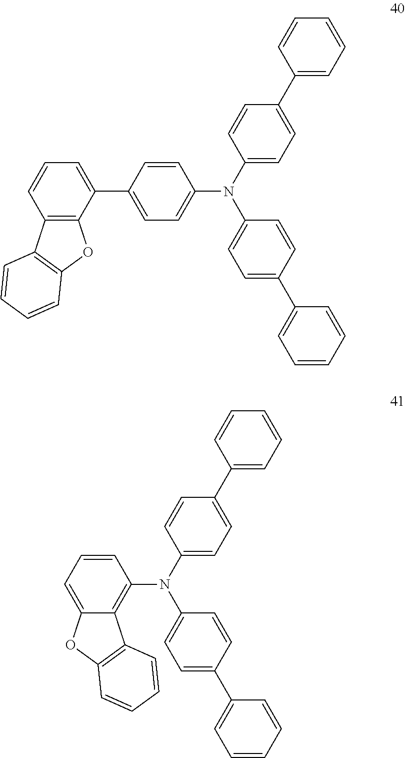 [SMILES: c1ccc(-c2ccc(N(c3ccc(-c4ccccc4)cc3)c3ccc(-c4cccc5c4oc4ccccc45)cc3)cc2)cc1 ; c1ccc(-c2ccc(N(c3ccc(-c4ccccc4)cc3)c3cccc4oc5ccccc5c34)cc2)cc1]

In Examples 1 to 6 and Comparative Examples 1 to 5, the substrate 102 was formed using a transparent glass substrate, the positive electrode 104 was formed using ITO to a thickness of about 150 nm, the hole injection layer 106 was formed using 2-TNATA to a thickness of about 60 nm, the hole transport layer 108 was formed using the respective compounds of the examples or the comparative examples to a thickness of about 30 nm, the emission layer 110 was formed using ADN doped with 3% TBP to a thickness of about 25 nm, the electron transport layer 112 was formed using Alq₃ to a thickness of about 25 nm, the electron injection layer 114 was formed using LiF to a thickness of about 1 nm, and the negative electrode 116 was formed using Al to a thickness of about 100 nm.

With respect to the organic EL devices thus manufactured, the voltage and the emission efficiency were evaluated. The evaluation was conducted at a current density of 10 mA/cm².

TABLE 1

| Device | Hole | Current | | Emission |
|---|---|---|---|---|
| manufacturing | transport | density | Voltage | efficiency |
| Example | material | mA/cm² | (V) | (cd/A) |
| Example 1 | Compound 3 | 10 | 5.5 | 9.6 |
| Example 2 | Compound 7 | 10 | 4.9 | 9.4 |
| Example 3 | Compound | 10 | 5.2 | 8.4 |
| | 17 | | | |
| Example 4 | Compound | 10 | 5.7 | 7.9 |
| | 21 | | | |
| Example 5 | Compound | 10 | 5.0 | 8.9 |
| | 26 | | | |
| Example 6 | Compound | 10 | 5.5 | 8.8 |
| | 33 | | | |
| Comparative | Compound | 10 | 7.5 | 5.2 |
| Example 1 | 37 | | | |
| Comparative | Compound | 10 | 8.1 | 6.3 |
| Example 2 | 38 | | | |
| Comparative | Compound | 10 | 8.1 | 8.2 |
| Example 3 | 39 | | | |
| Comparative | Compound | 10 | 7.5 | 8.0 |
| Example 4 | 40 | | | |
| Comparative | Compound | 10 | 8.0 | 8.4 |
| Example 5 | 41 | | | |

From the results shown in Table 1, the organic EL devices in which amine derivatives including a 1-dibenzofuran group and a 1-substituted dibenzofuran part, as the materials for an organic EL device according to Examples 1 to 6 were applied in hole transport layers, were proved to be driven at a lower voltage and have higher emission efficiency when compared to the compounds according to the comparative examples. The results were thought to be obtainable because the energy gap (e.g., the triplet energy gap) of the 1-substituted dibenzofuran derivative was increased, energy transfer to an adjacent layer was restrained, and the introduction of electrons from an emission layer was blocked in the materials for an organic EL device according to Examples 1 to 6, but the present disclosure is not limited to any particular mechanism or theory.

Meanwhile, the driving voltage was high and the emission efficiency was lowered in the amine derivatives having a 2-substituted dibenzofuran part according to the comparative examples. For example, since a dibenzofuran part and an amine part form a conjugated structure in the material for an organic EL device according to Comparative Example 2, radical stability during transporting carriers is thought to be deteriorated, but the present disclosure is not limited by any particular mechanism or theory. While the emission efficiency was high, the driving voltage was decreased if an amine derivative of Compound 39 having a 3-substituted dibenzofuran part and an amine derivative of Compound 40 having a 4-substituted dibenzofuran part according to Comparative Examples 3 and 4 were applied.

It was proved that equal or higher emission efficiency as that of Comparative Examples 3 and 4 and a low driving voltage could be realized by using the amine derivative of Compound 3 having a 1-substituted dibenzofuran part according to Example 1. It could be thought that carrier mobility may be improved further, and suitable or appropriate energy gap in an organic EL device may be obtained for the 1-substituted dibenzofuran derivative rather than the 3-substituted dibenzofuran derivative, but the present disclosure is not limited by any particular mechanism or theory.

In addition, in the case that a 1-dibenzofuran group was introduced without using a connecting group in an amine as in Comparative Example 5, high emission efficiency and high voltage were obtained, and thus, the connecting group was suggested as an effective means for lowering a voltage. The connecting group was proved to result in the decrease of the voltage in an amine derivative having a heteroarylene group as shown in Example 6 as well as an unsubstituted arylene group.

Since a material for an organic EL device according to an embodiment of the present disclosure uses an amine derivative having 1-dibenzofuran group and 1-substituted dibenzofuran part, energy gap (e.g., a triplet energy gap) may increase, energy transfer to an adjacent layer may be restrained, and the driving at a low voltage and high emission efficiency may be realized.

According to the present disclosure, a material for an organic EL device capable of being driven at a low voltage and having high emission efficiency, and an organic EL device including the same may be provided. For example, according to embodiments of the present disclosure, a material for an organic EL device capable of being driven at a low voltage and having high emission efficiency, for example, in a blue emission region and a green emission region, which is used in an emission layer or at least one layer of stacking layers disposed between the emission layer and an anode, and an organic EL device including the same may be provided. In embodiments of the present disclosure, an amine derivative having a 1-dibenzofuran group and a 1-substituted dibenzofuran part is used, and energy gap (e.g., a triplet energy gap) may increase, energy transfer to an adjacent layer may be restrained, and the driving at a low voltage and high emission efficiency may be realized.

As used herein, the terms “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present disclosure refers to “one or more embodiments of the present disclosure.” As used herein, the terms “use,” “using,” and “used” may be considered synonymous with the terms “utilize,” “utilizing,” and “utilized,” respectively. Also, the term “exemplary” is intended to refer to an example or illustration.

The terminology used herein is for the purpose of describing particular embodiments only and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, acts, operations, elements, and/or components, but do not preclude the presence or addition of one or more other features, integers, acts, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present.

Spatially relative terms, such as “beneath,” “below,” “lower,” “under,” “above,” “upper,” and the like, may be used herein for ease of explanation to describe one element or feature's relationship to another element(s) or feature(s) as illustrated in the FIGURES. It will be understood that the spatially relative terms are intended to encompass different orientations of the device in use or in operation, in addition to the orientation depicted in the FIGURES. For example, if the device in the FIGURES is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

The above-disclosed subject matter is to be considered illustrative and not restrictive, and the appended claims are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent device represented by Formula 1: 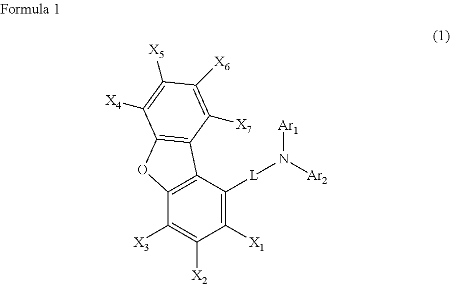
where X₁-X₇ are each independently a hydrogen atom, a deuterium atom, a halogen atom, an alkyl group having 1 to 15 carbon atoms, a substituted or unsubstituted aryl group having 6 to 30 carbon atoms for forming a ring, or a substituted or unsubstituted heteroaryl group having 5 to 30 carbon atoms for forming a ring,
Ar₁ and Ar₂ are each an aryl group having 6 to 30 carbon atoms for forming a ring, or a heteroaryl group having 1 to 30 carbon atoms for forming a ring,
each of Ar₁ and Ar₂ does not have the same structure as a dibenzofuran group including L in Formula 1, and
L is a divalent connecting group having a triplet energy gap of 2.5 eV or above.

2. The material for an organic electroluminescent device of claim 1, wherein L is a divalent group selected from a substituted or unsubstituted arylene group represented by Formula 2 and a substituted or unsubstituted heteroarylene group in which at least one ring carbon of Formula 2 is substituted with a heteroatom, and wherein n is an integer from 1 to 3:

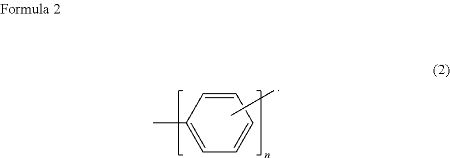

3. An organic electroluminescent device, comprising:
a material for an organic electroluminescent device in an emission layer,
wherein the material is represented by Formula 1: 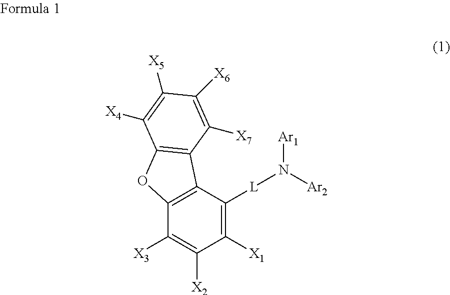
where X₁-X₇ are each independently a hydrogen atom, a deuterium atom, a halogen atom, an alkyl group having 1 to 15 carbon atoms, a substituted or unsubstituted aryl group having 6 to 30 carbon atoms for forming a ring, or a substituted or unsubstituted heteroaryl group having 5 to 30 carbon atoms for forming a ring,
Ar₁ and Ar₂ are each an aryl group having 6 to 30 carbon atoms for forming a ring, or a heteroaryl group having 1 to 30 carbon atoms for forming a ring, and
each of Ar₁ and Ar₂ does not have the same structure as a dibenzofuran group including L in Formula 1, and
L is a divalent connecting group having a triplet energy gap of 2.5 eV or above.

4. The organic electroluminescent device of claim 3, wherein L is a divalent group selected from a substituted or unsubstituted arylene group represented by Formula 2 and a substituted or unsubstituted heteroarylene group in which at least one ring carbon of Formula 2 is substituted with a heteroatom, and wherein n is an integer from 1 to 3:

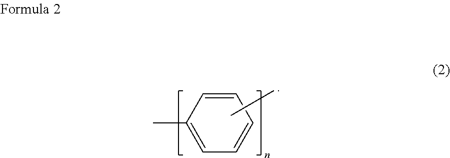

5. The organic electroluminescent device of claim 4, wherein the material comprises at least one selected from Compounds 1 to 36:

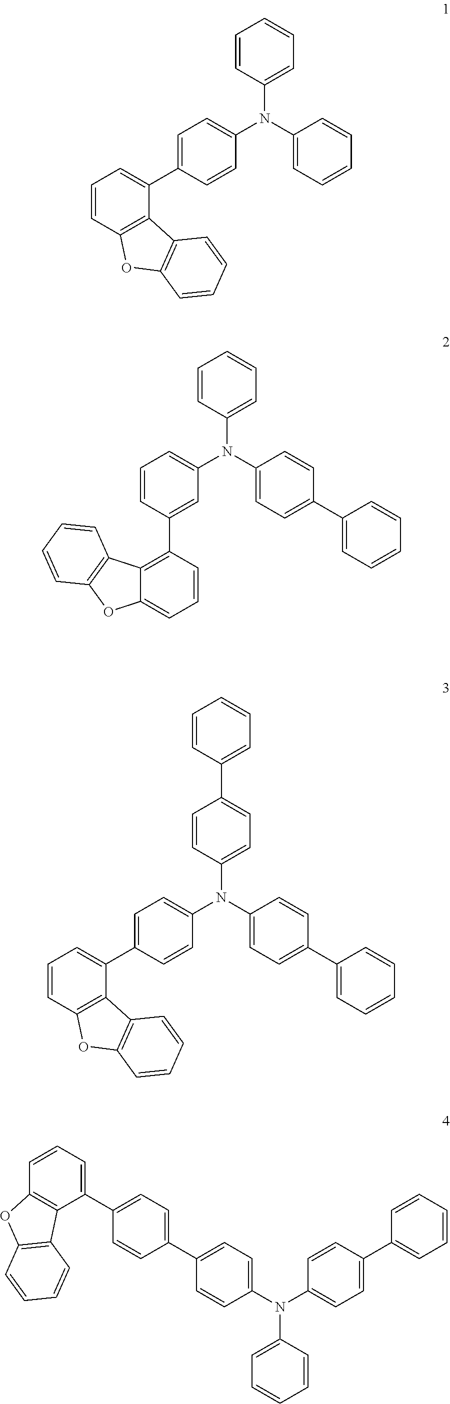 [SMILES: c1ccc(N(c2ccccc2)c2ccc(-c3cccc4oc5ccccc5c34)cc2)cc1 ; c1ccc(-c2ccc(N(c3ccccc3)c3cccc(-c4cccc5oc6ccccc6c45)c3)cc2)cc1 ; c1ccc(-c2ccc(N(c3ccc(-c4ccccc4)cc3)c3ccc(-c4cccc5oc6ccccc6c45)cc3)cc2)cc1 ; c1ccc(-c2ccc(N(c3ccccc3)c3ccc(-c4ccc(-c5cccc6oc7ccccc7c56)cc4)cc3)cc2)cc1]

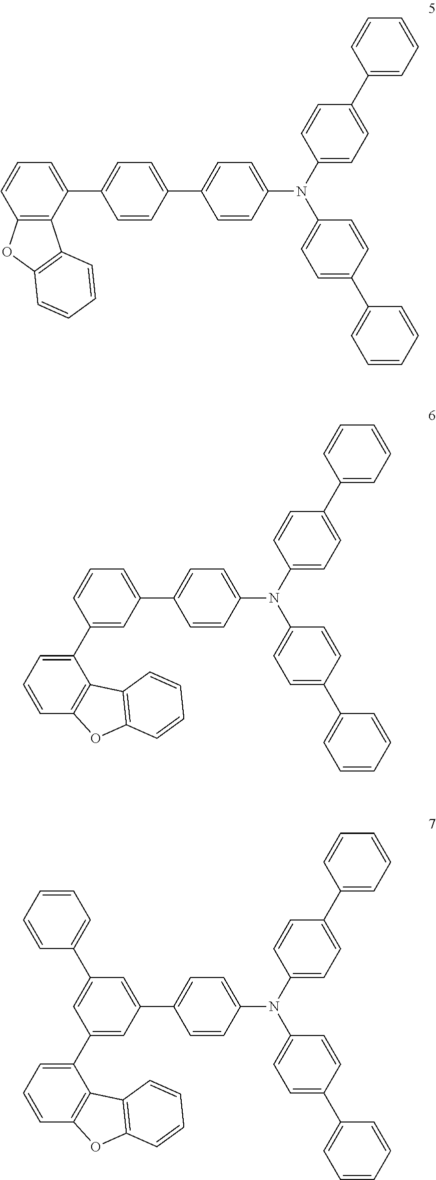 [SMILES: c1ccc(-c2ccc(N(c3ccc(-c4ccccc4)cc3)c3ccc(-c4ccc(-c5cccc6oc7ccccc7c56)cc4)cc3)cc2)cc1 ; c1ccc(-c2ccc(N(c3ccc(-c4ccccc4)cc3)c3ccc(-c4cccc(-c5cccc6oc7ccccc7c56)c4)cc3)cc2)cc1 ; c1ccc(-c2ccc(N(c3ccc(-c4ccccc4)cc3)c3ccc(-c4cc(-c5ccccc5)cc(-c5cccc6oc7ccccc7c56)c4)cc3)cc2)cc1]

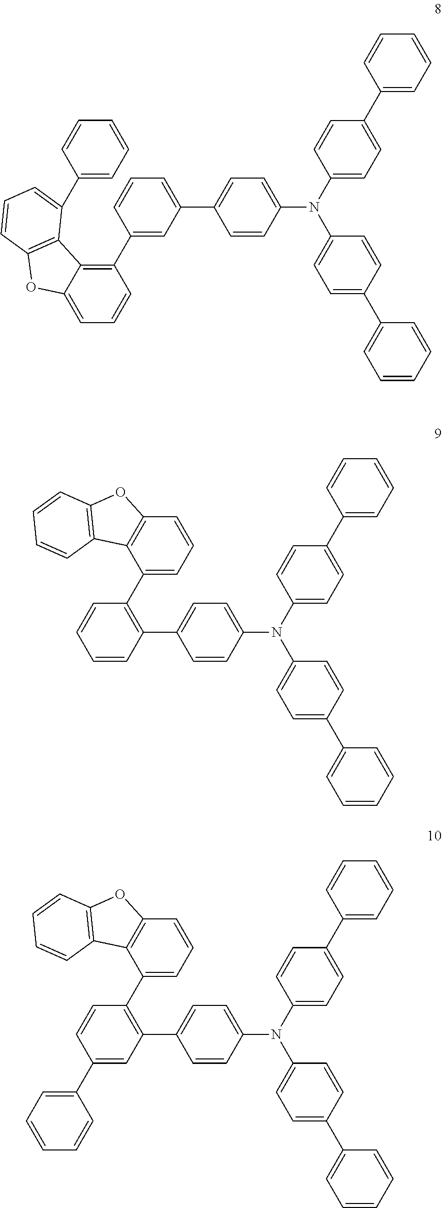 [SMILES: c1ccc(-c2ccc(N(c3ccc(-c4ccccc4)cc3)c3ccc(-c4cccc(-c5cccc6oc7cccc(-c8ccccc8)c7c56)c4)cc3)cc2)cc1 ; c1ccc(-c2ccc(N(c3ccc(-c4ccccc4)cc3)c3ccc(-c4ccccc4-c4cccc5oc6ccccc6c45)cc3)cc2)cc1 ; c1ccc(-c2ccc(N(c3ccc(-c4ccccc4)cc3)c3ccc(-c4cc(-c5ccccc5)ccc4-c4cccc5oc6ccccc6c45)cc3)cc2)cc1]

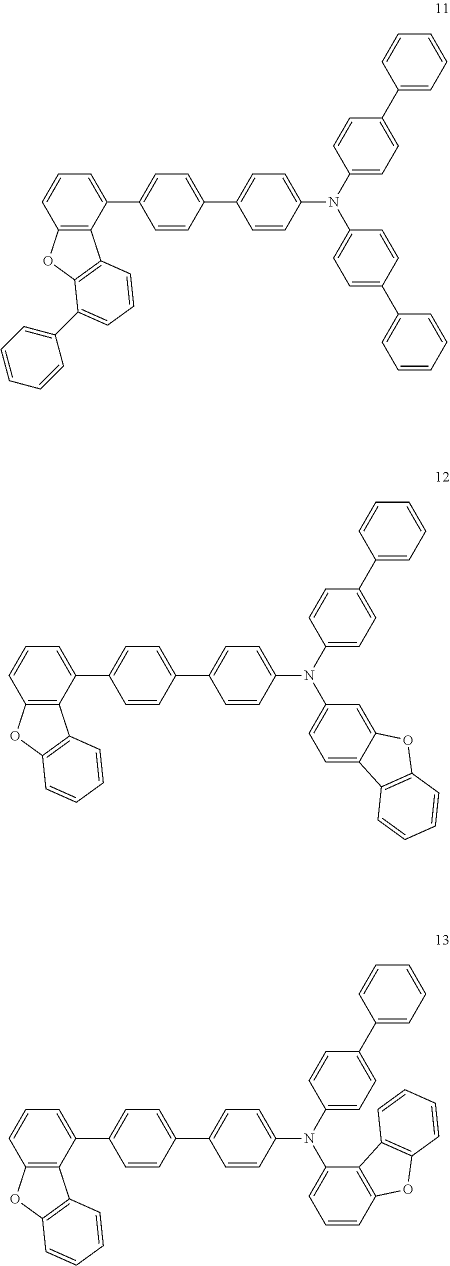 [SMILES: c1ccc(-c2ccc(N(c3ccc(-c4ccccc4)cc3)c3ccc(-c4ccc(-c5cccc6oc7c(-c8ccccc8)cccc7c56)cc4)cc3)cc2)cc1 ; c1ccc(-c2ccc(N(c3ccc(-c4ccc(-c5cccc6oc7ccccc7c56)cc4)cc3)c3ccc4c(c3)oc3ccccc34)cc2)cc1 ; c1ccc(-c2ccc(N(c3ccc(-c4ccc(-c5cccc6oc7ccccc7c56)cc4)cc3)c3cccc4oc5ccccc5c34)cc2)cc1]

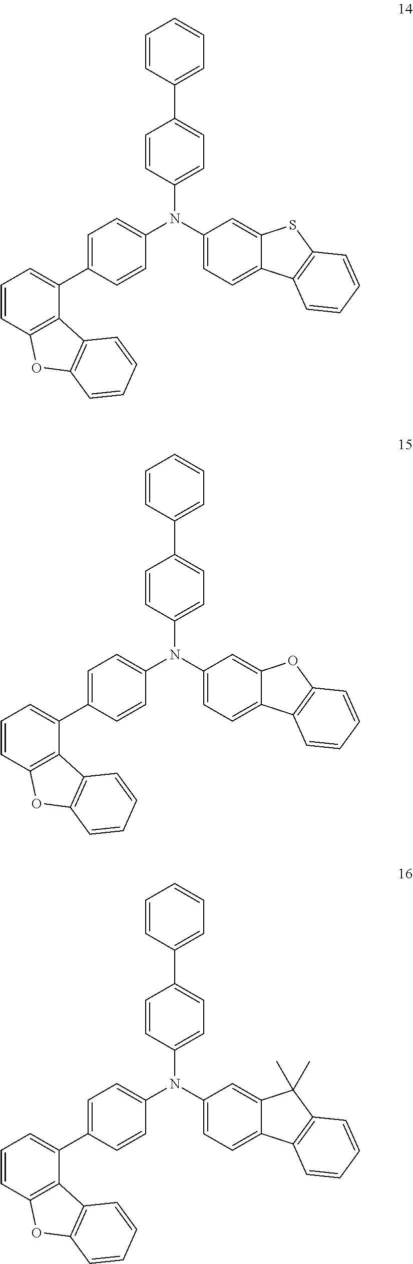 [SMILES: c1ccc(-c2ccc(N(c3ccc(-c4cccc5oc6ccccc6c45)cc3)c3ccc4c(c3)sc3ccccc34)cc2)cc1 ; c1ccc(-c2ccc(N(c3ccc(-c4cccc5oc6ccccc6c45)cc3)c3ccc4c(c3)oc3ccccc34)cc2)cc1 ; CC1(C)c2ccccc2-c2ccc(N(c3ccc(-c4ccccc4)cc3)c3ccc(-c4cccc5oc6ccccc6c45)cc3)cc21]

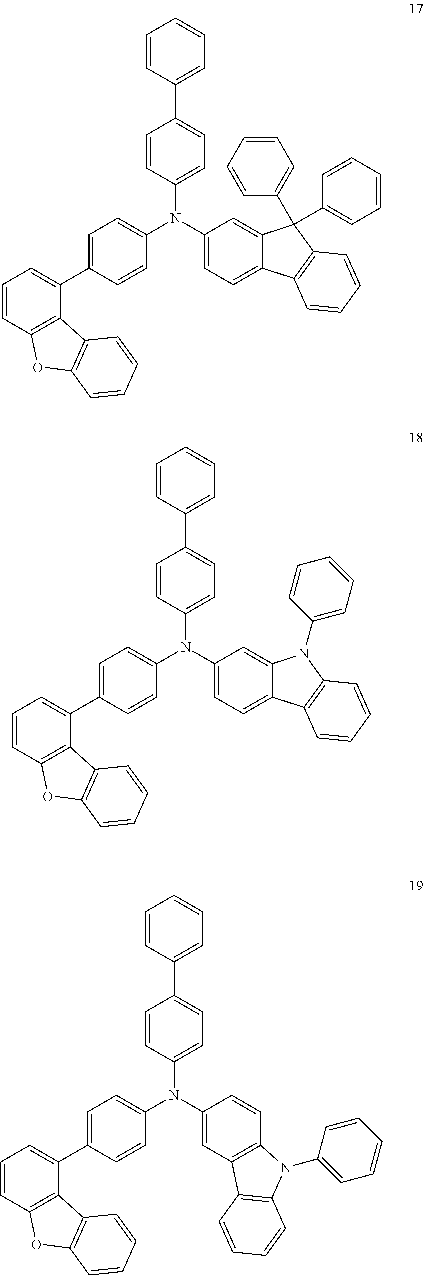 [SMILES: c1ccc(-c2ccc(N(c3ccc(-c4cccc5oc6ccccc6c45)cc3)c3ccc4c(c3)C(c3ccccc3)(c3ccccc3)c3ccccc3-4)cc2)cc1 ; c1ccc(-c2ccc(N(c3ccc(-c4cccc5oc6ccccc6c45)cc3)c3ccc4c5ccccc5n(-c5ccccc5)c4c3)cc2)cc1 ; c1ccc(-c2ccc(N(c3ccc(-c4cccc5oc6ccccc6c45)cc3)c3ccc4c(c3)c3ccccc3n4-c3ccccc3)cc2)cc1]

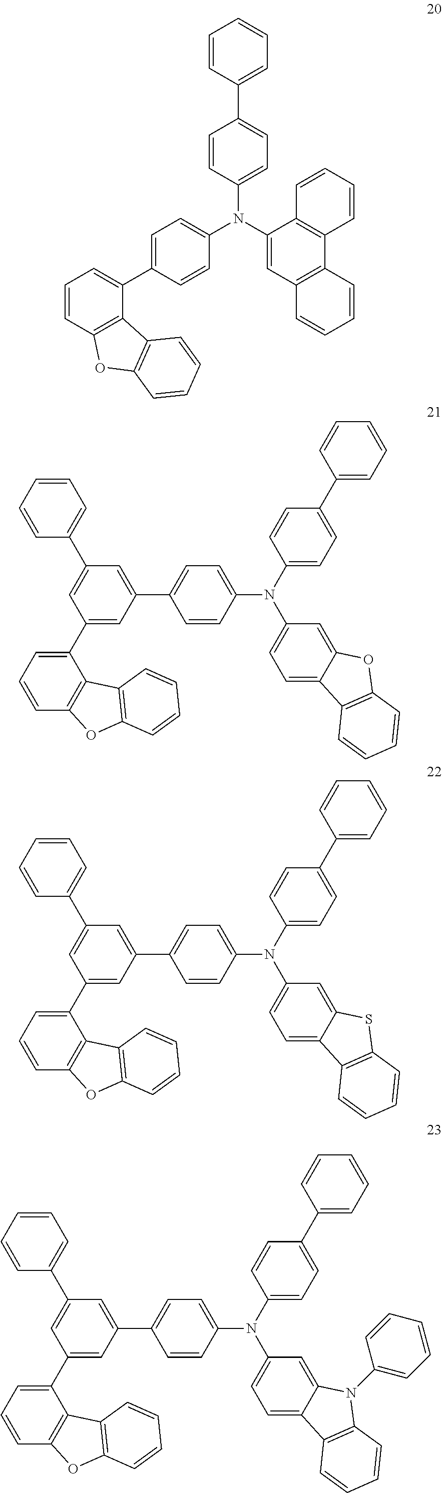

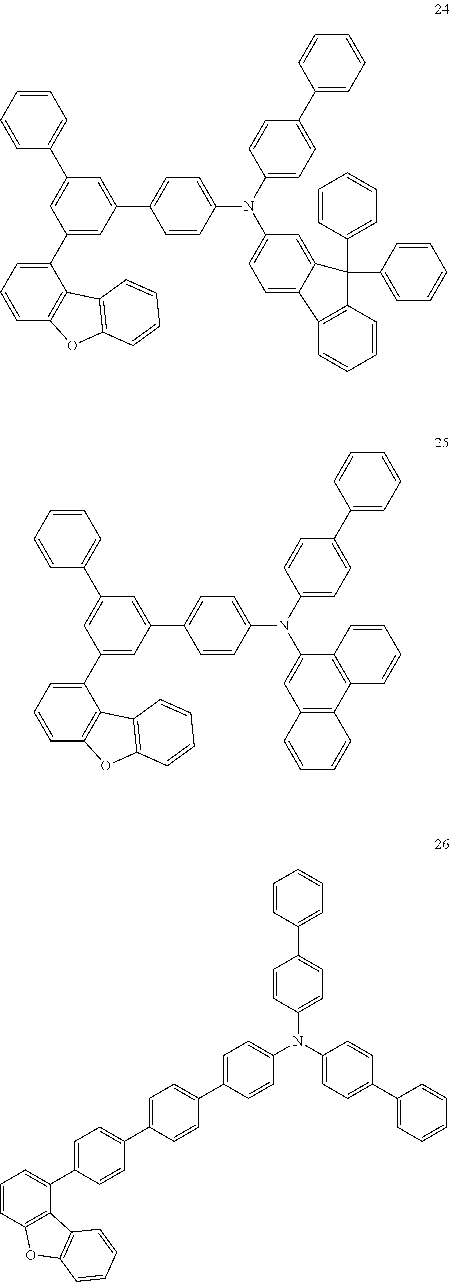 [SMILES: c1ccc(-c2ccc(N(c3ccc(-c4cc(-c5ccccc5)cc(-c5cccc6oc7ccccc7c56)c4)cc3)c3ccc4c(c3)C(c3ccccc3)(c3ccccc3)c3ccccc3-4)cc2)cc1 ; c1ccc(-c2ccc(N(c3ccc(-c4cc(-c5ccccc5)cc(-c5cccc6oc7ccccc7c56)c4)cc3)c3cc4ccccc4c4ccccc34)cc2)cc1 ; c1ccc(-c2ccc(N(c3ccc(-c4ccccc4)cc3)c3ccc(-c4ccc(-c5ccc(-c6cccc7oc8ccccc8c67)cc5)cc4)cc3)cc2)cc1]

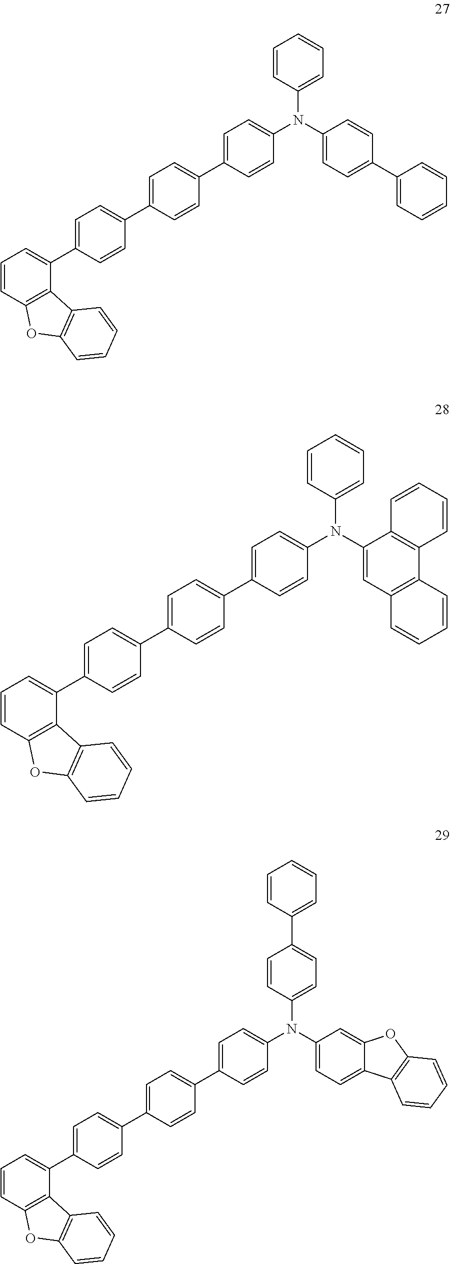 [SMILES: c1ccc(-c2ccc(N(c3ccccc3)c3ccc(-c4ccc(-c5ccc(-c6cccc7oc8ccccc8c67)cc5)cc4)cc3)cc2)cc1 ; c1ccc(N(c2ccc(-c3ccc(-c4ccc(-c5cccc6oc7ccccc7c56)cc4)cc3)cc2)c2cc3ccccc3c3ccccc23)cc1 ; c1ccc(-c2ccc(N(c3ccc(-c4ccc(-c5ccc(-c6cccc7oc8ccccc8c67)cc5)cc4)cc3)c3ccc4c(c3)oc3ccccc34)cc2)cc1]

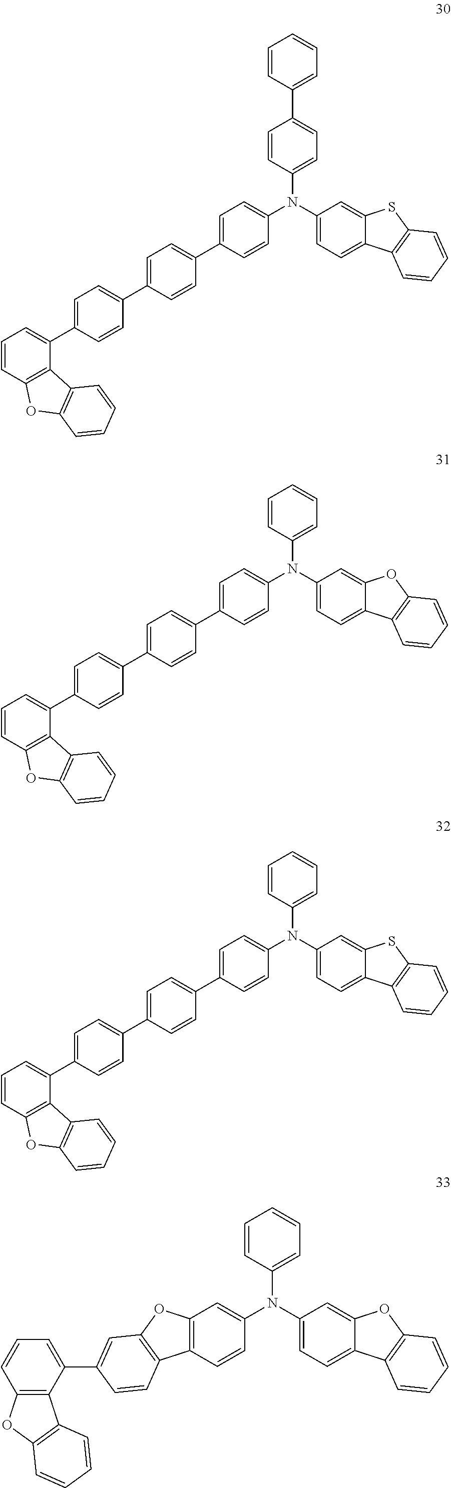 [SMILES: c1ccc(-c2ccc(N(c3ccc(-c4ccc(-c5ccc(-c6cccc7oc8ccccc8c67)cc5)cc4)cc3)c3ccc4c(c3)sc3ccccc34)cc2)cc1 ; c1ccc(N(c2ccc(-c3ccc(-c4ccc(-c5cccc6oc7ccccc7c56)cc4)cc3)cc2)c2ccc3c(c2)oc2ccccc23)cc1 ; c1ccc(N(c2ccc(-c3ccc(-c4ccc(-c5cccc6oc7ccccc7c56)cc4)cc3)cc2)c2ccc3c(c2)sc2ccccc23)cc1 ; c1ccc(N(c2ccc3c(c2)oc2ccccc23)c2ccc3c(c2)oc2cc(-c4cccc5oc6ccccc6c45)ccc23)cc1]

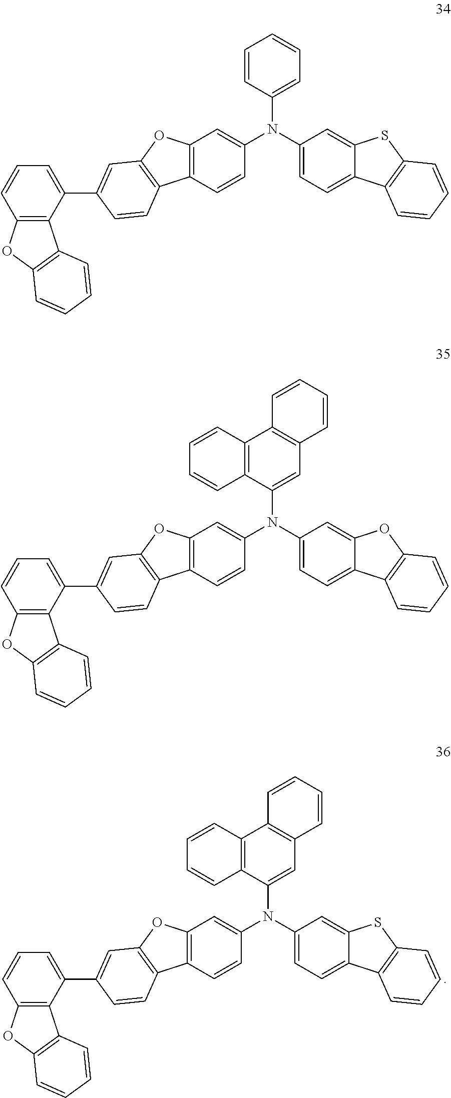 [SMILES: c1ccc(N(c2ccc3c(c2)oc2cc(-c4cccc5oc6ccccc6c45)ccc23)c2ccc3c(c2)sc2ccccc23)cc1 ; c1ccc2c(c1)cc(N(c1ccc3c(c1)oc1ccccc13)c1ccc3c(c1)oc1cc(-c4cccc5oc6ccccc6c45)ccc13)c1ccccc12 ; c1ccc2c(c1)cc(N(c1ccc3c(c1)oc1cc(-c4cccc5oc6ccccc6c45)ccc13)c1ccc3c(c1)sc1ccccc13)c1ccccc12]

6. An organic electroluminescent device, comprising:
a material in at least one layer of stacking layers between an emission layer and an anode,
wherein the material device is represented by Formula 1: 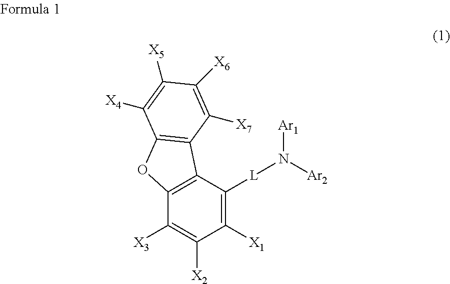
wherein X₁-X₇ are each independently a hydrogen atom, a deuterium atom, a halogen atom, an alkyl group having 1 to 15 carbon atoms, a substituted or unsubstituted aryl group having 6 to 30 carbon atoms for forming a ring, or a substituted or unsubstituted heteroaryl group having 5 to 30 carbon atoms for forming a ring,
Ar₁ and Ar₂ are each an aryl group having 6 to 30 carbon atoms for forming a ring, or a heteroaryl group having 1 to 30 carbon atoms for forming a ring, and
each of Ar₁ and Ar₂ does not have the same structure as a dibenzofuran group including L in Formula 1, and
L is a divalent connecting group having a triplet energy gap of 2.5 eV or above.

7. The organic electroluminescent device of claim 6, wherein L is a divalent group selected from a substituted or unsubstituted arylene group represented by Formula 2 and a substituted or unsubstituted heteroarylene group in which at least one ring carbon of Formula 2 is substituted with a heteroatom, and wherein n is an integer from 1 to 3:

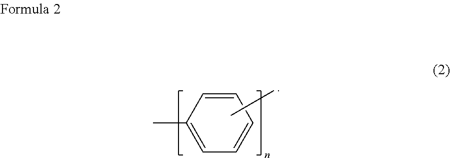

8. The organic electroluminescent device of claim 7, wherein the material for comprises at least one selected from Compounds 1 to 36: 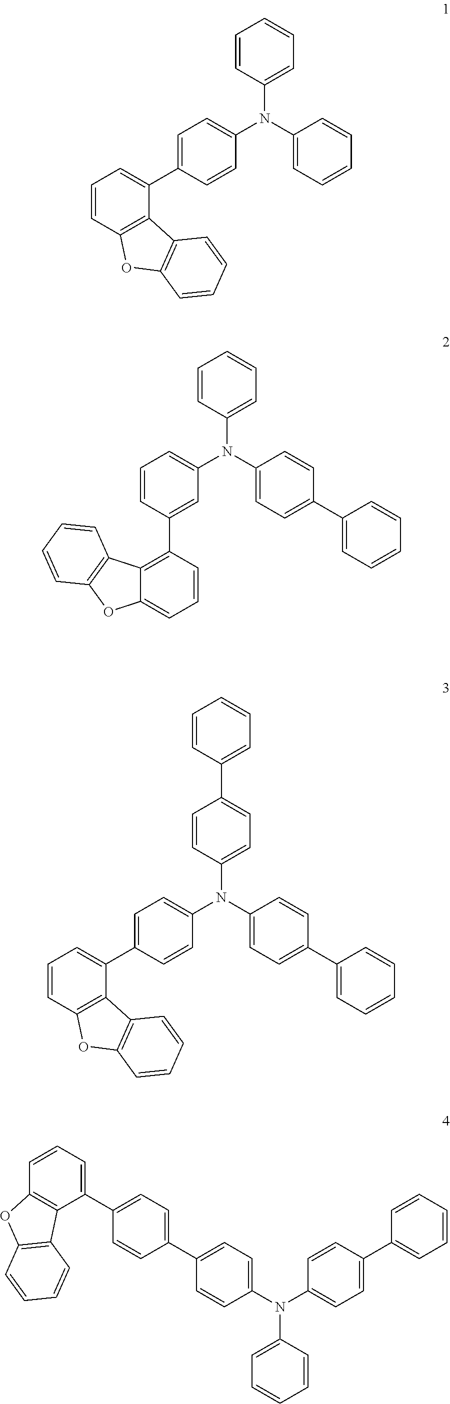 [SMILES: c1ccc(N(c2ccccc2)c2ccc(-c3cccc4oc5ccccc5c34)cc2)cc1 ; c1ccc(-c2ccc(N(c3ccccc3)c3cccc(-c4cccc5oc6ccccc6c45)c3)cc2)cc1 ; c1ccc(-c2ccc(N(c3ccc(-c4ccccc4)cc3)c3ccc(-c4cccc5oc6ccccc6c45)cc3)cc2)cc1 ; c1ccc(-c2ccc(N(c3ccccc3)c3ccc(-c4ccc(-c5cccc6oc7ccccc7c56)cc4)cc3)cc2)cc1] 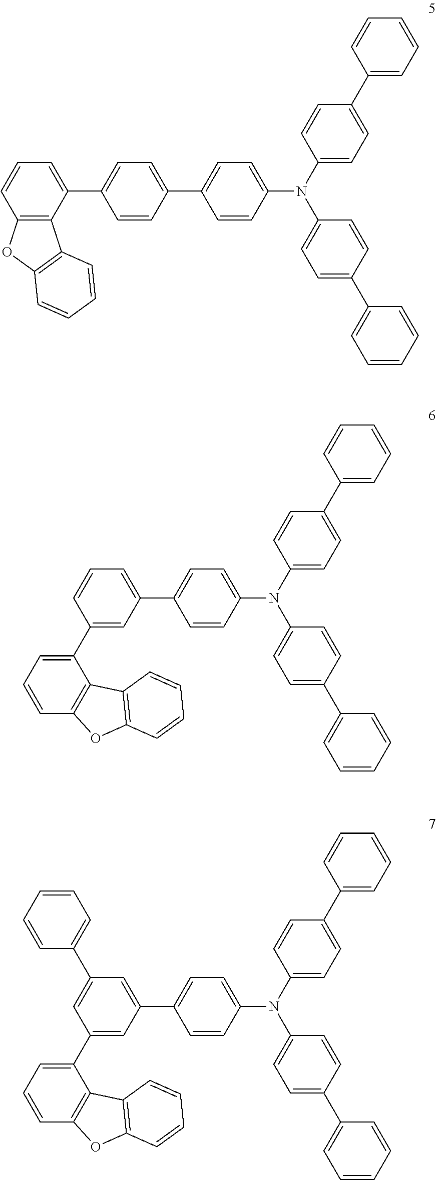 [SMILES: c1ccc(-c2ccc(N(c3ccc(-c4ccccc4)cc3)c3ccc(-c4ccc(-c5cccc6oc7ccccc7c56)cc4)cc3)cc2)cc1 ; c1ccc(-c2ccc(N(c3ccc(-c4ccccc4)cc3)c3ccc(-c4cccc(-c5cccc6oc7ccccc7c56)c4)cc3)cc2)cc1 ; c1ccc(-c2ccc(N(c3ccc(-c4ccccc4)cc3)c3ccc(-c4cc(-c5ccccc5)cc(-c5cccc6oc7ccccc7c56)c4)cc3)cc2)cc1] 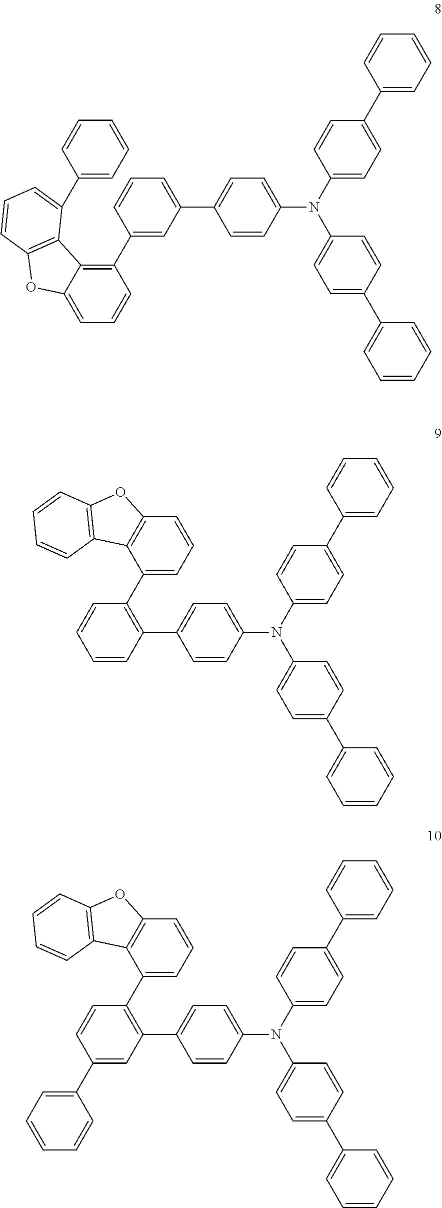 [SMILES: c1ccc(-c2ccc(N(c3ccc(-c4ccccc4)cc3)c3ccc(-c4cccc(-c5cccc6oc7cccc(-c8ccccc8)c7c56)c4)cc3)cc2)cc1 ; c1ccc(-c2ccc(N(c3ccc(-c4ccccc4)cc3)c3ccc(-c4ccccc4-c4cccc5oc6ccccc6c45)cc3)cc2)cc1 ; c1ccc(-c2ccc(N(c3ccc(-c4ccccc4)cc3)c3ccc(-c4cc(-c5ccccc5)ccc4-c4cccc5oc6ccccc6c45)cc3)cc2)cc1] 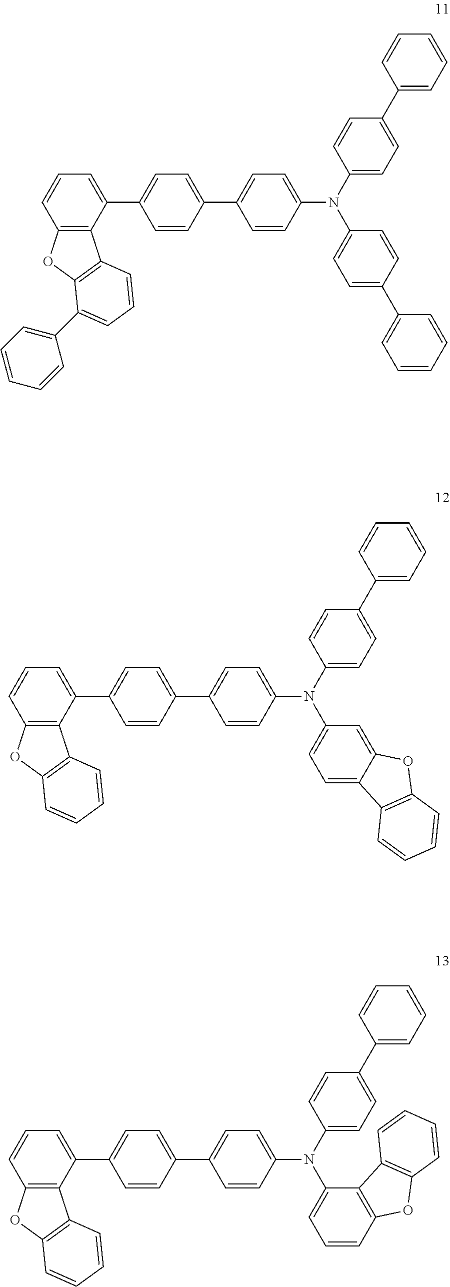 [SMILES: c1ccc(-c2ccc(N(c3ccc(-c4ccccc4)cc3)c3ccc(-c4ccc(-c5cccc6oc7c(-c8ccccc8)cccc7c56)cc4)cc3)cc2)cc1 ; c1ccc(-c2ccc(N(c3ccc(-c4ccc(-c5cccc6oc7ccccc7c56)cc4)cc3)c3ccc4c(c3)oc3ccccc34)cc2)cc1 ; c1ccc(-c2ccc(N(c3ccc(-c4ccc(-c5cccc6oc7ccccc7c56)cc4)cc3)c3cccc4oc5ccccc5c34)cc2)cc1] 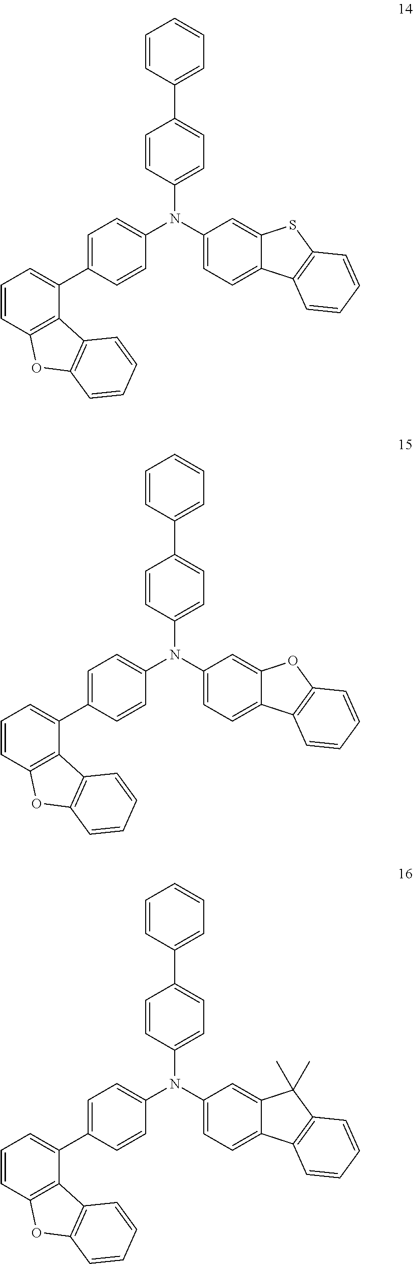 [SMILES: c1ccc(-c2ccc(N(c3ccc(-c4cccc5oc6ccccc6c45)cc3)c3ccc4c(c3)sc3ccccc34)cc2)cc1 ; c1ccc(-c2ccc(N(c3ccc(-c4cccc5oc6ccccc6c45)cc3)c3ccc4c(c3)oc3ccccc34)cc2)cc1 ; CC1(C)c2ccccc2-c2ccc(N(c3ccc(-c4ccccc4)cc3)c3ccc(-c4cccc5oc6ccccc6c45)cc3)cc21] 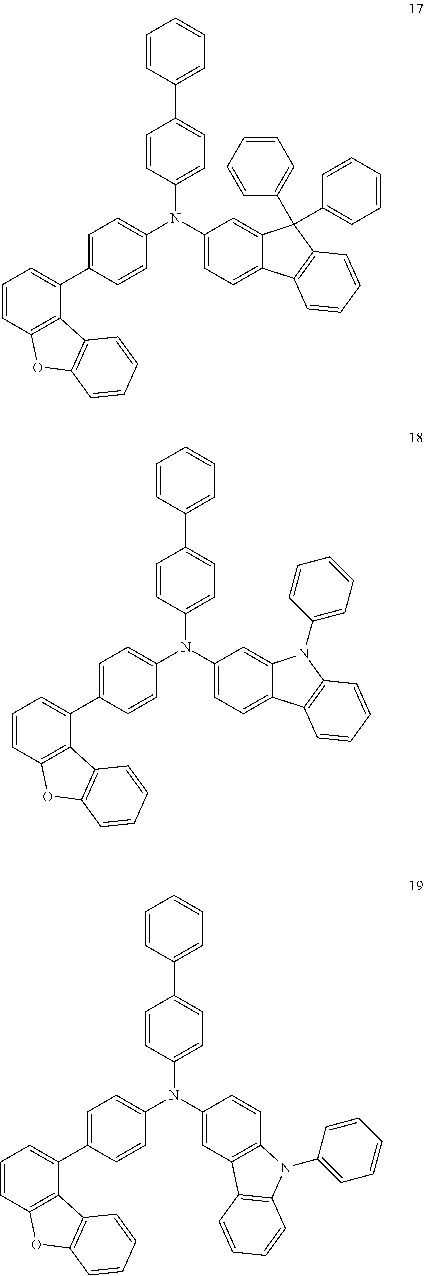 [SMILES: c1ccc(-c2ccc(N(c3ccc(-c4cccc5oc6ccccc6c45)cc3)c3ccc4c(c3)C(c3ccccc3)(c3ccccc3)c3ccccc3-4)cc2)cc1 ; c1ccc(-c2ccc(N(c3ccc(-c4cccc5oc6ccccc6c45)cc3)c3ccc4c5ccccc5n(-c5ccccc5)c4c3)cc2)cc1 ; c1ccc(-c2ccc(N(c3ccc(-c4cccc5oc6ccccc6c45)cc3)c3ccc4c(c3)c3ccccc3n4-c3ccccc3)cc2)cc1] 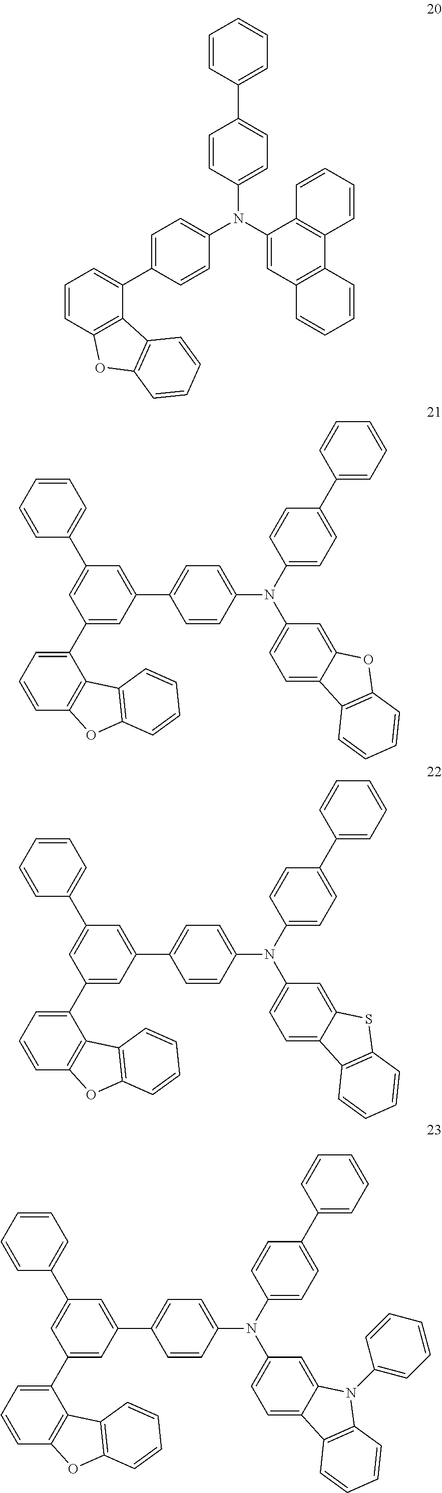 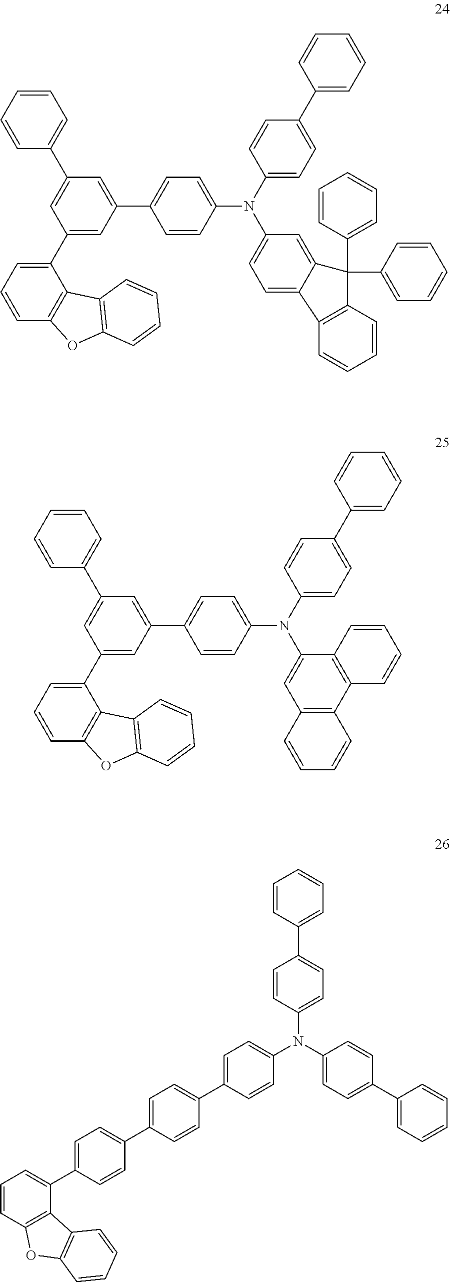 [SMILES: c1ccc(-c2ccc(N(c3ccc(-c4cc(-c5ccccc5)cc(-c5cccc6oc7ccccc7c56)c4)cc3)c3ccc4c(c3)C(c3ccccc3)(c3ccccc3)c3ccccc3-4)cc2)cc1 ; c1ccc(-c2ccc(N(c3ccc(-c4cc(-c5ccccc5)cc(-c5cccc6oc7ccccc7c56)c4)cc3)c3cc4ccccc4c4ccccc34)cc2)cc1 ; c1ccc(-c2ccc(N(c3ccc(-c4ccccc4)cc3)c3ccc(-c4ccc(-c5ccc(-c6cccc7oc8ccccc8c67)cc5)cc4)cc3)cc2)cc1] 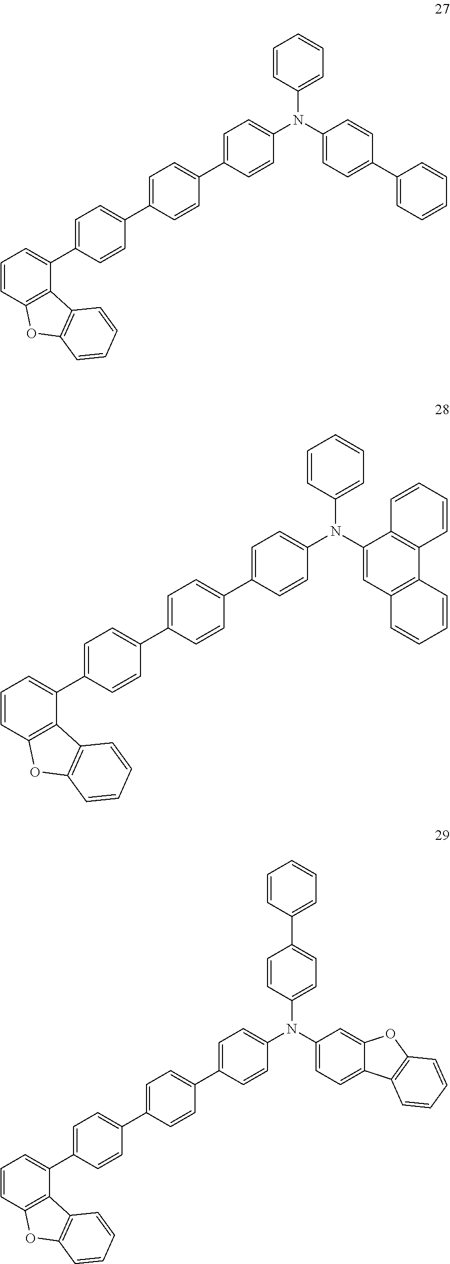 [SMILES: c1ccc(-c2ccc(N(c3ccccc3)c3ccc(-c4ccc(-c5ccc(-c6cccc7oc8ccccc8c67)cc5)cc4)cc3)cc2)cc1 ; c1ccc(N(c2ccc(-c3ccc(-c4ccc(-c5cccc6oc7ccccc7c56)cc4)cc3)cc2)c2cc3ccccc3c3ccccc23)cc1 ; c1ccc(-c2ccc(N(c3ccc(-c4ccc(-c5ccc(-c6cccc7oc8ccccc8c67)cc5)cc4)cc3)c3ccc4c(c3)oc3ccccc34)cc2)cc1] 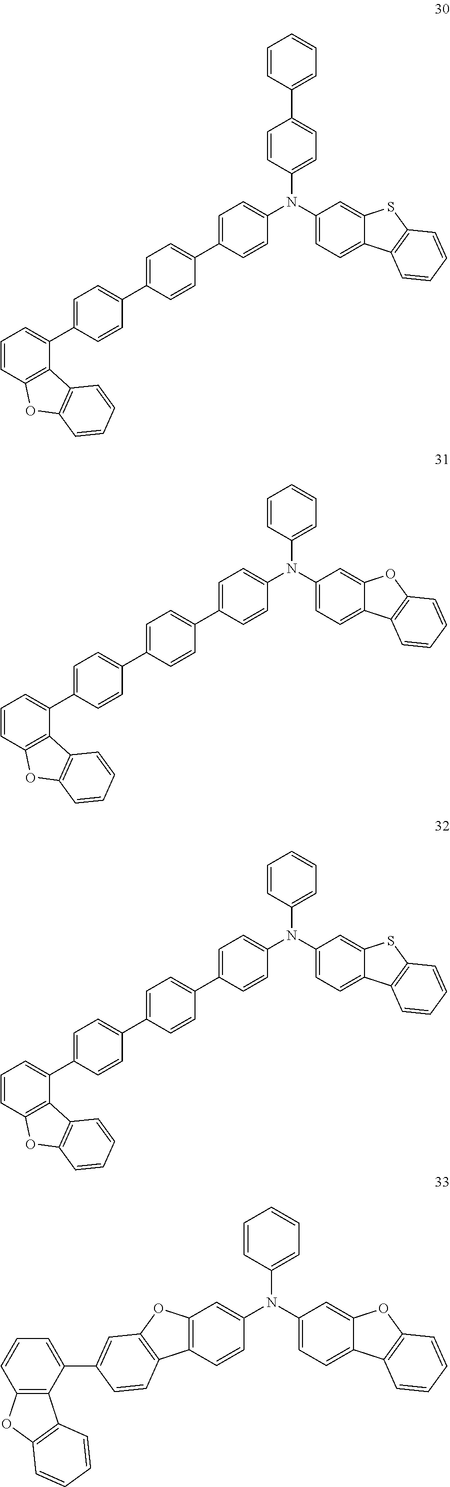 [SMILES: c1ccc(-c2ccc(N(c3ccc(-c4ccc(-c5ccc(-c6cccc7oc8ccccc8c67)cc5)cc4)cc3)c3ccc4c(c3)sc3ccccc34)cc2)cc1 ; c1ccc(N(c2ccc(-c3ccc(-c4ccc(-c5cccc6oc7ccccc7c56)cc4)cc3)cc2)c2ccc3c(c2)oc2ccccc23)cc1 ; c1ccc(N(c2ccc(-c3ccc(-c4ccc(-c5cccc6oc7ccccc7c56)cc4)cc3)cc2)c2ccc3c(c2)sc2ccccc23)cc1 ; c1ccc(N(c2ccc3c(c2)oc2ccccc23)c2ccc3c(c2)oc2cc(-c4cccc5oc6ccccc6c45)ccc23)cc1] 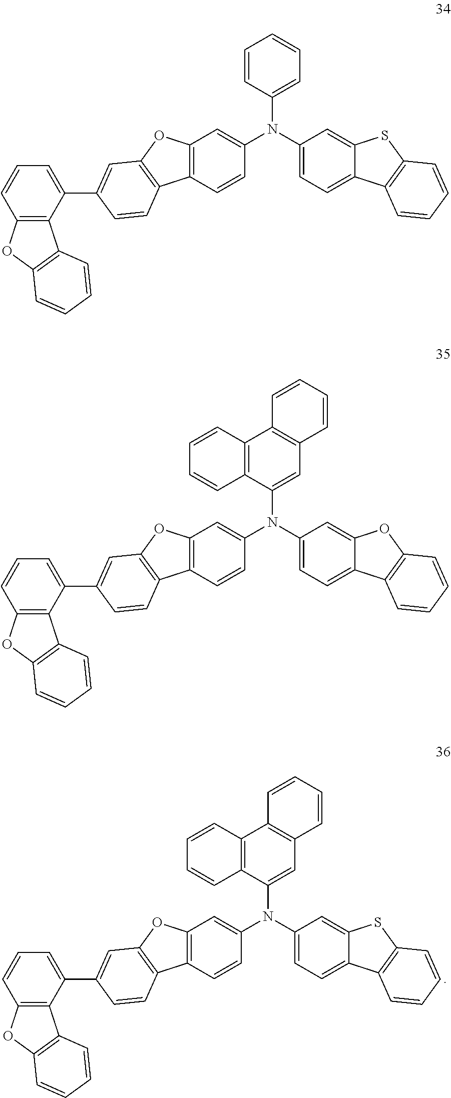 [SMILES: c1ccc(N(c2ccc3c(c2)oc2cc(-c4cccc5oc6ccccc6c45)ccc23)c2ccc3c(c2)sc2ccccc23)cc1 ; c1ccc2c(c1)cc(N(c1ccc3c(c1)oc1ccccc13)c1ccc3c(c1)oc1cc(-c4cccc5oc6ccccc6c45)ccc13)c1ccccc12 ; c1ccc2c(c1)cc(N(c1ccc3c(c1)oc1cc(-c4cccc5oc6ccccc6c45)ccc13)c1ccc3c(c1)sc1ccccc13)c1ccccc12]
